(19) United States
(12) Patent Application Publication
(10) Pub. No.: US 2018/0016493 A1
(43) Pub. Date: Jan. 18, 2018

(54) BLUE FLUORESCENT EMITTERS

(71) Applicant: Technische Universitaet Dresden, Dresden (DE)

(72) Inventors: Ramunas Lygaitis, Dresden (DE); Reinhard Scholz, Freital (DE); Olaf Zeika, Dresden (DE); Sebastian Reineke, Dresden (DE); Karl Leo, Dresden (DE); Simone Hofmann, Dresden (DE); Martin Oberlaender, Dresden (DE)

(21) Appl. No.: 15/548,774

(22) PCT Filed: Feb. 4, 2016
(86) PCT No.: PCT/EP2016/052413

§ 371 Date: Aug. 4, 2017

(30) Foreign Application Priority Data
Feb. 6, 2015 (DE) 10 2015 101 767.9

(51) Int. Cl. C09K 11/06; C07D 209/86; C07D 215/06; C07D 219/02; C07D 223/22; C07D 235/18; C07D 265/36; H01L 51/00; C07D 519/00; C07D 209/08; C07D 401/14; C07D 279/22; C07D 279/16; C07D 265/38; C07D 487/04; H01L 51/50

(52) U.S. Cl. CPC C09K 11/06; C07D 487/04; C07D 209/86; C07D 215/06; C07D 219/02; C07D 223/22; C07D 235/18; C07D 265/36; H01L 51/0072; H01L 51/0071; H01L 51/0067; C07D 519/00; C07D 209/08; C07D 401/14; C07D 279/22; C07D 279/16; C07D 265/38; H01L 51/001; C09K 2211/1007; C09K 2211/1014; C09K 2211/1022; H01L 51/0097; H01L 51/5012; H01L 2251/5338

(57) ABSTRACT

The present invention relates to compounds of the formula (I)

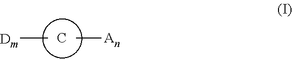

where C, D, A, m and n are as defined herein, and to their use in optoelectronic components and to optoelectronic components comprising them.

### CROSS-REFERENCE TO RELATED APPLICATIONS

The present application is a national stage entry according to 35 U.S.C. §371 of PCT Application No. PCT/EP2016/052413 filed on Feb. 4, 2016, which claims priority to German Patent Application No. 10 2015 101 767.9, filed on Feb. 6, 2015; both of which are herein incorporated by reference in their entirety.

### TECHNICAL FIELD

The subject matter herein generally provides compounds of formula (I) as defined herein, as well as their use as emitter or carrier material in an optoelectronic component.

### BACKGROUND

The development of novel functional compounds for use in electronic devices is currently the subject of intensive research. The aim here is the development and study of compounds which have not been used to date in electronic devices, and the development of compounds which enable an improved profile of properties of the devices.

According to a non-limiting embodiment, the term “optoelectronic component” is understood to mean inter alia organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-laser) and organic electroluminescent devices (OLEDs).

The construction of organic electroluminescent devices (OLEDs), in which the compounds described herein can be used as functional materials, is known to the skilled person and is described inter alia in patent publications U.S. Pat. No. 4,539,507, U.S. Pat. No. 5,151,629, EP 0676461 and WO 1998/27136.

In relation to the performance data of the OLEDs, especially with regard to broad commercial use, further improvements are still required. Of particular significance in this connection are the lifetime, efficiency and operating voltage of the OLEDs, and the color values achieved. Particularly in the case of blue-emitting OLEDs, there is potential for improvement with regard to the lifetime of the devices. In addition, it is desirable for the compounds for use as functional materials in electronic devices to have high thermal stability and a high glass transition temperature and to be sublimable without decomposing. In relation to the performance data of the OLEDs, especially with regard to broad commercial use, further improvements are still required. Of particular significance in this connection are the lifetime, efficiency and operating voltage of the OLEDs, and the color values achieved and the color rendering index.

Particularly in industry, there is an urgent need for long-lived, efficient blue emitters for OLEDs which, on top of that, are also producible inexpensively.

The excitons formed in the emitter layer in the course of recombination of holes and electrons are divided in a ratio of 1:3 between the singlet and triplet states. The use of organometallic complex structures benefits from the triplet excitons that are otherwise lost to electroluminescence via non-radiative quenching processes. Especially matrix materials doped with iridium or other precious metals, which according to the prior art frequently contain carbazole derivatives, for example bis(carbazolyl)biphenyl, and also ketones (WO 2004/093207), phosphine oxides, sulfones (WO 2005/003253), triazine compounds such as triazinylspirobifluorene (WO 2005/053055 and WO 2010/05306), find use as phosphorescent emitters; in addition, metal complexes, for example bis(2-methyl-8-quinolinolato-N1,O8)-(1,1′-biphenyl-4-olato)aluminum (BAIq) or bis[2-(2-benzothiazole)-phenolate]zinc(11) are also used.

Owing to spin restrictions in the process of recombination of the electrons and holes in the emitter layer, it is consequently possible in fluorescent emitters for only a maximum of 25% of the electrical energy to be converted to light. In materials in which the triplet state is at high energy and is thus close to the singlet state, reverse intersystem crossing (RISC) is possible. This raises triplet excitons thermally to the singlet state, which is referred to as singlet harvesting. Thus, it is theoretically possible for up to 100% of the energy stored in the energetically excited states to be emitted in the form of fluorescent electroluminescence. Organic molecules composed of donor and acceptor structures where the highest occupied molecular orbital (HOMO) and lowest unoccupied molecular orbital (LUMO) overlap slightly constitute efficient and inexpensive alternatives to precious metal-doped organic matrix materials. More particularly, a remedy could be provided in this connection by p-electron-deficient cycles substituted by p-electron-rich nitrogen-containing heterocycles, by virtue of their intensive, thermally activated delayed fluorescence.

### SUMMARY

According to a non-limiting embodiment, a compound of the general formula (I) is presented, i.e.:

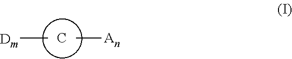

where C denotes an aromatic or heteroaromatic, nitrogen-containing 6-membered carbon ring selected from the group consisting of benzene, pyridine, pyrimidine, pyridazine, triazine and pyrazine;
the substituent D at each instance independently denotes a nitrogen-containing heteroaryl radical bonded to the aromatic system C via a nitrogen atom, or a radical of the formula —N(Ar)₂, —N(HetAr)(Ar), —N(HetAr)₂, where each Ar is independently an aryl radical and each HetAr is independently a heteroaryl radical;
the substituent A at each instance independently denotes electron acceptors selected from the group consisting of C1-C12 perfluoroalkyl, especially CF₃, Cl, F, Br, SCN or CN;
m denotes an integer selected from 1, 2, 3, 4 and 5;
n denotes an integer selected from 1, 2, 3, 4 and 5;
where 3≦m+n≦6,
and where the substituents D and A are each bonded to the aromatic system C,
wherein not more than one substituent A denotes CN.

### DETAILED DESCRIPTION

It has now been surprisingly found that, the compounds of the formula (I) are of excellent suitability for use in optoelectronic components, especially as emitter or matrix material. The compounds of the formula (I) are notable for their high-energy triplet states, as a result of which the lowest excited singlet state of the compounds of the formula (I) can be thermally populated via this triplet state, and so the efficiency in the energy conversion can theoretically reach values of up to 100%.

Formula (I) may have the following formula:

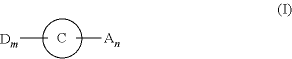

where

C denotes an aromatic or heteroaromatic, nitrogen-containing 6-membered carbon ring selected from the group consisting of benzene, pyridine, pyrimidine, pyridazine, triazine and pyrazine;

the substituent D at each instance independently denotes a nitrogen-containing heteroaryl radical bonded to the aromatic system C via a nitrogen atom, or a radical of the formula —N(Ar)2, —N(HetAr)(Ar), —N(HetAr)2, where each Ar is independently an aryl radical and each HetAr is independently a heteroaryl radical;

the substituent A at each instance independently denotes electron acceptors selected from the group consisting of C1-C12 perfluoroalkyl, especially CF3, CCl3, Cl, F, Br, SCN or CN;

m denotes an integer selected from 1, 2, 3, 4 and 5;

n denotes an integer selected from 1, 2, 3, 4 and 5;

where 3≦m+n≦6, especially 4≦m+n≦6,

and where the substituents D and A are each bonded to the aromatic system C,

with the proviso that not more than one substituent A denotes CN.

A further embodiment includes the use of at least one of the compounds described herein in an optoelectronic component, for example an electronic electroluminescent device (OLED), an organic integrated circuit (O-IC), an organic field-effect transistor (O-FET), an organic thin-film transistor (O-TFT), an organic light-emitting transistor (O-LET), an organic solar cell (O-SC), an organic optical detector, an organic photoreceptor, an organic field-quench device (O-FQD), a light-emitting electrochemical cell (LEC) or an organic laser diode (O-laser).

Finally, another non-limiting embodiment also relates to optoelectronic components such as those mentioned above which comprise at least one of the compounds described herein.

“At least one” as used herein means 1 or more, i.e. 1, 2, 3, 4, 5, 6, 7, 8, 9 or more. “At least one substituent” thus means, for example, at least one kind of substituent, which can mean one kind of substituent or a mixture of several different substituents.

The compounds the general structural formula (I) may have the following formula:

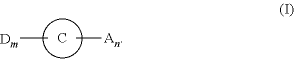

In this formula, the substituents D are nitrogen-containing, electron-rich, mono- or polycyclic heteroaryl rings or diaryl- or diheteroarylamines and the substituents A are electron acceptors in specific arrangements. The substituents D and A are bonded to an aromatic or N-heteroaromatic 6-membered carbon ring C. The indices m and n each denote an integer selected from 1, 2, 3, 4 or 5, where m+n is not more than 6, but not less than 3, i.e. 3≦m+n≦6, depending on the substitutable carbon atoms in the aromatic system C. The substituents D and A and the aromatic system C are described in detail hereinafter.

Thus, the substituent A at each instance is independently C1-C12 perfluoroalkyl, such as but not limited to CF3, Cl, F, Br, SCN or CN. The substituent D at each instance is independently a nitrogen-containing heteroaryl radical bonded to the aromatic system C via a nitrogen atom, or a radical of the formula —N(Ar)2, —N(HetAr)(Ar), —N(HetAr)2, where each Ar is independently an aryl radical and each HetAr is independently a heteroaryl radical. C denotes a correspondingly substituted benzene, pyridine, pyrimidine, pyridazine, triazine or pyrazine ring.

The indices m and n are dependent on one another and each denote an integer selected from 1, 2, 3, 4 and 5. The sum total of m and n here is not more than 6, but not less than 3, i.e. 3≦m+n≦6. If C, for example, denotes a benzene ring, a total of 6 positions in the aromatic system may be occupied by the substituents D and A in any combination, where D and A are each represented at least once. If C, for example, denotes a pyrimidine ring, it is possible for a total of 4 positions in the aromatic system to be occupied by the substituents D and A in any combination, where D and A are each represented at least once. In various embodiments, 4≦m+n≦6 or 4≦m+n≦5 or 5≦m+n≦6 or 3≦m+n≦4, especially 4≦m+n≦6.

In respect of compounds of the formula (I), there is the additional proviso that just one substituent A in the aromatic system C is CN.

Unless defined differently herein, an aryl group may contain from 6 to 60 aromatic ring atoms and a heteroaryl group may contain from 5 to 60 aromatic ring atoms, at least one of which is a heteroatom.

In addition, an aryl group or heteroaryl group may include either a monocyclic aromatic group, for example phenyl, or a monocyclic heteroaromatic group, for example pyridinyl, pyrimidinyl or thienyl, or a fused (annelated, polycyclic) aromatic or heteroaromatic polycyclic group, for example naphthalenyl, phenanthrenyl or carbazolyl. In the context of the present application, a fused (annelated, polycyclic) aromatic or heteroaromatic polycycle consists of two or more simple (monocyclic) aromatic or heteroaromatic rings fused to one another.

A nitrogen-containing heteroaryl (HetAr) may include an aromatic ring system containing at least one nitrogen atom and optionally further heteroatoms, such as but not limited to S and O. The nitrogen-containing heteroaryl is attached as electron-rich substituent D to the aromatic system C in the general structural formula (I) via the at least one nitrogen atom. The nitrogen-containing heteroaryl radicals may be substituted, where the substituents are selected, for example, from halogens, substituted or unsubstituted straight-chain alkyl, alkoxy or thioalkyl groups having 1 to 20 carbon atoms or substituted or unsubstituted, branched or cyclic alkyl, alkoxy or thioalkyl groups having 3 to 20 carbon atoms or substituted or unsubstituted alkenyl or alkynyl groups having 2 to 20 carbon atoms, where each of the aforementioned groups may include one or more heteroatoms selected from Si, O, S, Se, N and P. Substituents for the nitrogen-containing heteroaryl radicals may be substituted or unsubstituted methyl, ethyl, propyl, isopropyl, c-propyl, n-butyl, sec-butyl, isobutyl and t-butyl groups and their corresponding alkoxy and thioalkyl equivalents. If the aforementioned substituents of the nitrogen-containing heteroaryl radicals are substituted in turn, the substituents thereof are selected from straight-chain alkyl, alkoxy or thioalkyl groups having 1 to 20 carbon atoms, cyclic or branched alkyl, alkoxy or thioalkyl groups having 3 to 20 carbon atoms, alkenyl or alkynyl groups having 2 to 20 carbon atoms, carboxyl, hydroxyl, thiol, amino, hydrazine or nitro groups, aryl or heteroaryl groups as defined below, and halogens, especially fluorine, and pseudohalogens (—CN, —N3, —OCN, —NCO, —CNO, —SCN, —NCS, —SeCN).

A nitrogen-containing heteroaryl may be substituted by further radicals and may include groups selected or derived from pyrrolyl, indolyl, isoindolyl, carbazolyl, pyridinyl, quinolinyl, isoquinolinyl, acridinyl, phenanthridinyl, benzo-5,6-quinolinyl, benzo-6,7-quinolinyl, benzo-7,8-quinolinyl, phenothiazinyl, phenoxazinyl, pyrazolyl, indazolyl, imidazolyl, benzimidazolyl, naphthimidazolyl, phenanthrimidazolyl, pyridimidazolyl, pyrazinimidazolyl, quinoxalinimidazolyl, oxazolyl, benzoxazolyl, naphthoxazolyl, anthroxazolyl, phenanthroxazolyl, isoxazolyl, 1,2-thiazolyl, 1,3-thiazolyl, benzothiazolyl, pyridazinyl, benzopyridazinyl, pyrimidinyl, benzpyrimidinyl, quinoxalinyl, pyrazinyl, phenazinyl, naphthyridinyl, carbazolyl, benzocarbolinyl, phenanthrolinyl, purinyl, pteridinyl and indolizinyl.

The formula —N(Ar)2 denotes a diarylamine bonded to the aromatic system C in the general formula (I) via the nitrogen atom, and which bears two aromatic substituents (Ar). The two aryl substituents may be identical or different. In various embodiments, they are selected from the aromatic groups specified below. By contrast with heteroaryl radicals, the two aromatic substituents Ar of the diarylamine radical —N(Ar)2 do not include any heteroatoms in their aromatic ring structures. However, they may be substituted, in which case the substituents may be the substituents described above in connection with the nitrogen-containing heteroaryl radicals.

An aryl (Ar) group may be substituted in each case by further radicals, such as but not limited to phenyl, naphthyl, anthracenyl, phenanthrenyl, fluorenyl, pyrenyl, dihydropyrenyl, chrysenyl, perylenyl, fluoranthenyl, benzanthracenyl, benzphenanthrenyl, tetracenyl, pentacenyl and benzpyrenyl, where the aforementioned groups may each be substituted or unsubstituted. If they are substituted, the substituents may be the substituents described above in connection with the nitrogen-containing heteroaryl radicals. In addition, aryl groups may also be bridged to one another via their substituents. Examples of corresponding bridged diarylamines are iminodibenzyls (10,11-dihydrobenzazepines) or 9H-acridine, but they may be understood to include phenoxazines and phenothiazines.

The formula —N(HetAr)(Ar) denotes an amine substituted by an aryl group and a heteroaryl group, and bonded to the aromatic system C in the general formula (I) via the nitrogen atom. The aryl group is as defined above. The heteroaryl group denotes an aromatic ring system which includes at least one heteroatom, such as but not limited to N, S and O, and which may additionally be substituted, where the substituents may be the substituents described above in connection with the nitrogen-containing heteroaryl radicals.

Analogously, the formula —N(HetAr)2 denotes an amine which bears to heteroaryl substituents and which is bonded to the aromatic system C in the general structural formula (I) via the nitrogen atom. The heteroaryl groups may be identical or different and are as defined above. More particularly, they may also be substituted, in which case the substituents may be the substituents described above in connection with the nitrogen-containing heteroaryl radicals.

A heteroaryl (HetAr) group may be substituted by further radicals in each case and may be joined to the aromatic system via any desired position and may be or include a heteroaryl, such as furanyl, difuranyl, terfuranyl, benzofuranyl, isobenzofuranyl, dibenzofuranyl, thienyl, dithienyl, terthienyl, benzothienyl, isobenzothienyl, benzodithienyl, benzotrithienyl, pyrrolyl, indolyl, isoindolyl, carbazolyl, pyridinyl, quinolinyl, isoquinolinyl, acridinyl, phenanthridinyl, benzo-5,6-quinolinyl, benzo-6,7-quinolinyl, benzo-7,8-quinolinyl, phenothiazinyl, phenoxazinyl, pyrazolyl, indazolyl, imidazolyl, benzimidazolyl, naphthimidazolyl, phenanthrimidazolyl, pyridimidazolyl, pyrazinimidazolyl, quinoxalinimidazolyl, oxazolyl, benzoxazolyl, naphthoxazolyl, anthroxazolyl, phenanthroxazolyl, isoxazolyl, 1,2-thiazolyl, 1,3-thiazolyl, benzothiazolyl, pyridazinyl, benzopyridazinyl, pyrimidinyl, benzpyrimidinyl, quinoxalinyl, pyrazinyl, phenazinyl, naphthyridinyl, carbazolyl, benzocarbolinyl, phenanthrolinyl, phenoxazines, phenothiazines, iminostilbenes, purinyl, pteridinyl and indolizinyl, and also fused systems of the above with one another and/or with aryl groups, for example naphthyl, anthracenyl, phenanthrenyl, fluorenyl, pyrenyl, dihydropyrenyl, chrysenyl, perylenyl, fluoranthenyl, benzanthracenyl, benzphenanthrenyl, tetracenyl, pentacenyl and benzpyrenyl, where the aforementioned groups may each be substituted or unsubstituted. If they are substituted, the substituents may be the substituents described above in connection with the nitrogen-containing heteroaryl radicals. In addition, heteroaryl groups may also be bridged to one another via their substituents.

In various embodiments, the compounds of the general formula (I) are compounds of one of the formulae (Ia) to (VIc).

In various embodiments, the compound of the general formula (I) is a compound of the general formula (Ia)

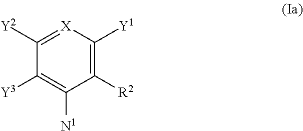

where

X is C—CF3, C—Cl, C—F, C—CN or N;

R² is F, Cl, Br, C1-C12 perfluoroalkyl, especially CF3, CN or H;

N1 is a nitrogen-containing heteroaryl radical bonded via a nitrogen atom, or a radical of the formula

—N(Ar)2, —N(HetAr)(Ar), —N(HetAr)2, where each Ar is independently an aryl radical and each HetAr is independently a heteroaryl radical;

Y1, Y2 and Y3 are each independently C1-C12 perfluoroalkyl, especially CF3, CCl3, F, Cl, Br, SCN, CN, H, a nitrogen-containing heteroaryl radical bonded via a nitrogen atom, or a radical of the formula —N(Ar)2, —N(HetAr)(Ar), —N(HetAr)2, where each Ar is independently an aryl radical and each HetAr is independently a heteroaryl radical;

with the proviso that

only one of the substituents Y1-3 and R2 is CN;

when X is C—CN, none of the substituents Y1-3 and R2 is CN.

when X is C—F, C—Cl or C—CN, and Y1 and Y2 are each independently a nitrogen-containing heteroaryl radical bonded via a nitrogen atom, or a radical of the formula —N(Ar)2, —N(HetAr)(Ar), —N(HetAr)2, where each Ar is independently an aryl radical and each HetAr is independently a heteroaryl radical, Y3 is not F, Cl, Br or CN.

In various embodiments, the compounds of the formula (Ia) are restricted to those compounds in which, when X is C—CN, N1 is a carbazole bonded via the nitrogen atom and Y1, Y2 and Y3 are each a carbazole bonded via the nitrogen atom, and R2 is not F.

In various embodiments of the compounds of the formula (Ia), the nitrogen-containing heteroaryl radical (N1) is pyrrolyl, indolyl, isoindolyl, carbazolyl, piperidinyl, tetrahydroquinolinyl, pyrrolidinyl, tetrahydroisoquinolinyl, acridinyl, phenanthridinyl, benzo-5,6-quinolinyl, benzomorpholinyl, benzothiomorpholinyl, benzo-6,7-quinolinyl, benzo-7,8-quinolinyl, phenothiazinyl, phenoxazinyl, pyrazolyl, indazolyl, imidazolyl, benzimidazolyl, naphthimidazolyl, phenanthrimidazolyl, pyridimidazolyl, pyrazinimidazolyl, quinoxalinimidazolyl, benzopyridazinyl, benzpyrimidinyl, quinoxalinyl, pyrazinyl, phenazinyl, naphthyridinyl, carbazolyl, benzocarbolinyl, purinyl, pteridinyl, or indolizinyl. The aforementioned may be substituted or unsubstituted, where the substituents of the aforementioned nitrogen-containing heteroaryl radicals are, for example, substituents selected from the group consisting of hydrogen, halogens, substituted or unsubstituted straight-chain alkyl, alkoxy or thioalkyl groups having 1 to 20 carbon atoms or substituted or unsubstituted, branched or cyclic alkyl, alkoxy or thioalkyl groups having 3 to 20 carbon atoms or substituted or unsubstituted alkenyl or alkynyl groups having 2 to 20 carbon atoms, where each of the aforementioned groups may include one or more heteroatoms selected from Si, O, S, Se, N and P. Assuming that the aforementioned substituents of the nitrogen-containing heteroaryl radicals are substituted in turn, the substituents thereof are selected from straight-chain alkyl, alkoxy or thioalkyl groups having 1 to 20 carbon atoms, cyclic or branched alkyl, alkoxy or thioalkyl groups having 3 to 20 carbon atoms, alkenyl or alkynyl groups having 2 to 20 carbon atoms, hydroxyl, thiol, amino, hydrazine, nitro or nitrile groups, unsubstituted aryl or heteroaryl groups as already defined, and halogens. Substituents of the nitrogen-containing heteroaryl radicals may further include hydrogen, methyl, ethyl, propyl, isopropyl, c-propyl, n-butyl, sec-butyl, isobutyl and t-butyl groups and their corresponding alkoxy and thioalkyl equivalents. The aforementioned nitrogen-containing heteroaryl radicals may additionally be fused to one or more aryl radical(s) or to one or more heteroaryl radical(s). Examples of such a fused system are dibenzazepine and 2,3-indolocarbazole. It is also possible for such fused aryl (heteroaryl) systems to be substituted or unsubstituted in turn. A non-limiting example of such a substituted fused system is 2,3-(1-methylindolo)carbazole.

In various embodiments of the compounds of the formula (Ia), the aryl radicals in the radicals of the formulae —N(Ar)2 and —N(HetAr)(Ar) are independently substituted or unsubstituted phenyl, naphthyl, anthracenyl, phenanthrenyl, fluorenyl, pyrenyl, dihydropyrenyl, chrysenyl, perylenyl, fluoranthenyl, benzanthracenyl, benzphenanthrenyl, tetracenyl, pentacenyl and benzpyrenyl. The substituents of the aforementioned aryl radicals are, for example, substituents selected from the group consisting of halogens, substituted or unsubstituted straight-chain alkyl, alkoxy or thioalkyl groups having 1 to 20 carbon atoms or substituted or unsubstituted, branched or cyclic alkyl, alkoxy or thioalkyl groups having 3 to 20 carbon atoms or substituted or unsubstituted alkenyl or alkynyl groups having 2 to 20 carbon atoms, where each of the aforementioned groups may include one or more heteroatoms selected from Si, O, S, Se, N and P. Assuming that the aforementioned substituents of the aryl radicals are in turn substituted, the substituents thereof are selected from straight-chain alkyl, alkoxy or thioalkyl groups having 1 to 20 carbon atoms, cyclic or branched alkyl, alkoxy or thioalkyl groups having 3 to 20 carbon atoms, alkenyl or alkynyl groups having 2 to 20 carbon atoms, hydroxyl, thiol, amino, hydrazine, nitro or nitrile groups, unsubstituted aryl or heteroaryl groups as already defined, and halogens. Substituents for the aryl radicals in the radicals of the formulae —N(Ar)2 and —N(HetAr)(Ar) are methyl, ethyl, propyl, isopropyl, c-propyl, n-butyl, sec-butyl, isobutyl and t-butyl groups and their corresponding alkoxy and thioalkyl equivalents. The aforementioned aryl radicals may additionally be fused to one or more aryl radical(s) of the aforementioned aryl radicals or to one or more heteroaryl radical(s). It is also possible for such fused aryl (heteroaryl) systems to be substituted or unsubstituted in turn. The aforementioned aryl radicals in the formulae —N(Ar)2 and —N(HetAr)(Ar) may also be bridged to one another or to the heteroaryl via their respective substituents. Non-limiting examples of such a bridged diarylamine are 9H-acridine and 10,11-dihydrobenzazepine. These bridged diarylamines of the formula —N(Ar)2 or aryl-heteroarylamines of the formula —N(HetAr)(Ar) may also be substituted or unsubstituted, substituents being as defined above. Some non-limiting examples of a substituted bridged diarylamine are 9,9-dimethylacridines, 9,9-dihydroacridines, carbazoles, iminodibenzyls, phenoxazines, phenothiazines, iminostilbenes (dibenzazepines), arylindolines, arylbenzomorpholines, arylbenzothiomorpholines or aryltetrahydroquinolines.

In various embodiments of the compounds of the formula (Ia), the heteroaryl radicals in the radicals of the formulae —N(HetAr)(Ar) and —N(HetAr)2 are independently substituted or unsubstituted furanyl, difuranyl, terfuranyl, benzofuranyl, isobenzofuranyl, dibenzofuranyl, thienyl, dithienyl, terthienyl, benzothienyl, isobenzothienyl, benzodithienyl, benzotrithienyl, pyrrolyl, indolyl, isoindolyl, carbazolyl, pyridinyl, quinolinyl, isoquinolinyl, acridinyl, phenanthridinyl, benzo-5,6-quinolinyl, benzo-6,7-quinolinyl, benzo-7,8-quinolinyl, phenothiazinyl, phenoxazinyl, pyrazolyl, indazolyl, imidazolyl, benzimidazolyl, naphthimidazolyl, phenanthrimidazolyl, pyridimidazolyl, pyrazinimidazolyl, quinoxalinimidazolyl, oxazolyl, benzoxazolyl, naphthoxazolyl, anthroxazolyl, phenanthroxazolyl, isoxazolyl, 1,2-thiazolyl, 1,3-thiazolyl, benzothiazolyl, pyridazinyl, benzopyridazinyl, pyrimidinyl, benzpyrimidinyl, quinoxalinyl, pyrazinyl, phenazinyl, naphthyridinyl, carbazolyl, benzocarbolinyl, phenanthrolinyl, 1,2,3-triazolyl, 1,2,4-triazolyl, benzotriazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, 1,3,5-triazinyl, 1,2,4-triazinyl, 1,2,3-triazinyl, tetrazolyl, 1,2,4,5-tetrazinyl, 1,2,3,4-tetrazinyl, 1,2,3,5-tetrazinyl, purinyl, pteridinyl, indolizinyl and benzothiadiazolyl. The substituents of the aforementioned heteroaryl radicals are, for example, substituents selected from the group consisting of halogens, substituted or unsubstituted straight-chain alkyl, alkoxy or thioalkyl groups having 1 to 20 carbon atoms or substituted or unsubstituted, branched or cyclic alkyl, alkoxy or thioalkyl groups having 3 to 20 carbon atoms or substituted or unsubstituted alkenyl or alkynyl groups having 2 to 20 carbon atoms, where each of the aforementioned groups may include one or more heteroatoms selected from Si, O, S, Se, N and P. Assuming that the aforementioned substituents of the heteroaryl radicals are substituted in turn, the substituents thereof are selected from straight-chain alkyl, alkoxy or thioalkyl groups having 1 to 20 carbon atoms, cyclic or branched alkyl, alkoxy or thioalkyl groups having 3 to 20 carbon atoms, alkenyl or alkynyl groups having 2 to 20 carbon atoms, hydroxyl, thiol, amino, hydrazine, nitro or nitrile groups, unsubstituted aryl or heteroaryl groups as already defined, and halogens. Non-limiting substituents of the heteroaryl radicals in the radicals of the formulae —N(HetAr)(Ar) and —N(HetAr)2 are methyl, ethyl, propyl, isopropyl, c-propyl, n-butyl, sec-butyl, isobutyl and t-butyl groups and their corresponding alkoxy and thioalkyl equivalents. The aforementioned heteroaryl radicals may additionally be fused to one or more heteroaryl radical(s) of the aforementioned heteroaryl radicals or to one or more aryl radical(s). It is also possible for such fused aryl (heteroaryl) systems to be substituted or unsubstituted in turn. The aforementioned heteroaryl radicals in the formulae —N(HetAr)(Ar) and —N(HetAr)2 may also be bridged to one another or to the aryl via their respective substituents. These bridged diarylamines of the formula —N(Ar)2 or aryl-heteroarylamines of the formula —N(HetAr)(Ar) may also be substituted or unsubstituted, where substituents are as defined above.

In further embodiments, the compound of the general formula (I) is a compound of the formula (II):

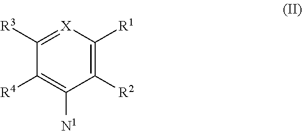

In the compound of the formula (II), X is C—CF3, C—CCl3, C—Cl, C—SCN or C—CN; N1 denotes a nitrogen-containing heteroaryl radical bonded via a nitrogen atom, or a radical of the formula —N(Ar)2,

—N(HetAr)(Ar), —N(HetAr)2, where each Ar is independently an aryl radical and each HetAr is independently a heteroaryl radical; and R1, R2, R3 and R4 each independently denote F, Cl, Br, CF3, SCN, CN or H, with the proviso that only one substituent R1-4 is CN, and that, when X is C—CN, none of the substituents R1-4 is CN.

In various embodiments of the compounds of the formula (II), the same selection criteria as defined above for compounds of the formula (Ia) are applicable to the nitrogen-containing heteroaryl radical (N1).

In various embodiments of the compounds of the formula (II), in addition, the same selection criteria as defined above for compounds of the formula (Ia) are applicable to aryl radicals in the radicals of the formulae —N(Ar)2 and —N(HetAr)(Ar) and to heteroaryl radicals in the radicals of the formulae —N(HetAr)(Ar) and —N(HetAr)2.

In various embodiments, the following definitions apply to the substituents of the compound of the formula (II):

The substituent X denotes C—CF3 or C—CN; the substituent N1 denotes a substituted or unsubstituted phenoxazine, phenothiazine, indole, benzimidazole, quinoline, dibenzazepine, carbazole or 9H-acridine radical, each of which is bonded to the aromatic system of the formula (II) via the nitrogen atom, such as a phenoxazine, phenothiazine, 2-phenylindoline, 2-phenylbenzimidazole, 2-naphthylbenzimidazole, 2-phenyl-1,2,3,4-tetrahydroquinoline, dibenzazepine, 10,11-dihydrobenzazepine-, 2,3-indolocarbazole, 2,3-(1-methylindolo)carbazole, 9H-acridine or 9,9-dimethylacridine radical, each of which is bonded to the aromatic system of the formula (II) via the nitrogen atom in the main carbon ring; the substituent R2 denotes CF3 or F; and the substituents R1, R3 and R4 denote F or H.

Specific embodiments of the compounds of the general formula (II) are the compounds 1a-1y and 4F1Cz:

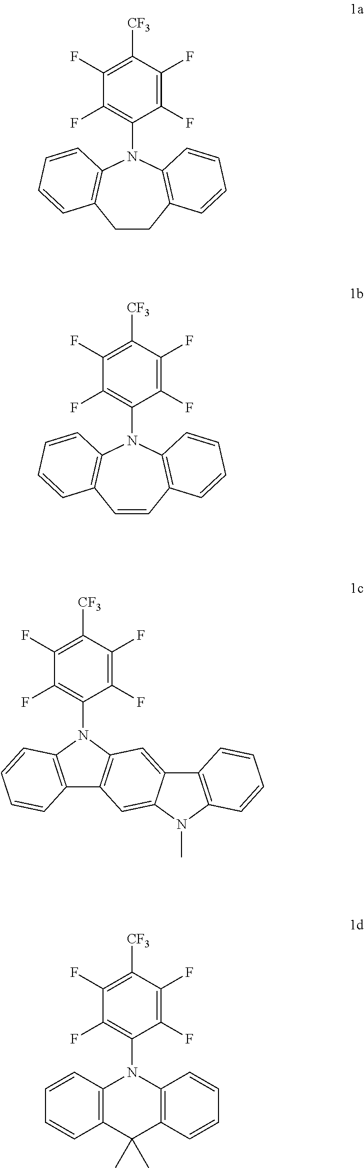

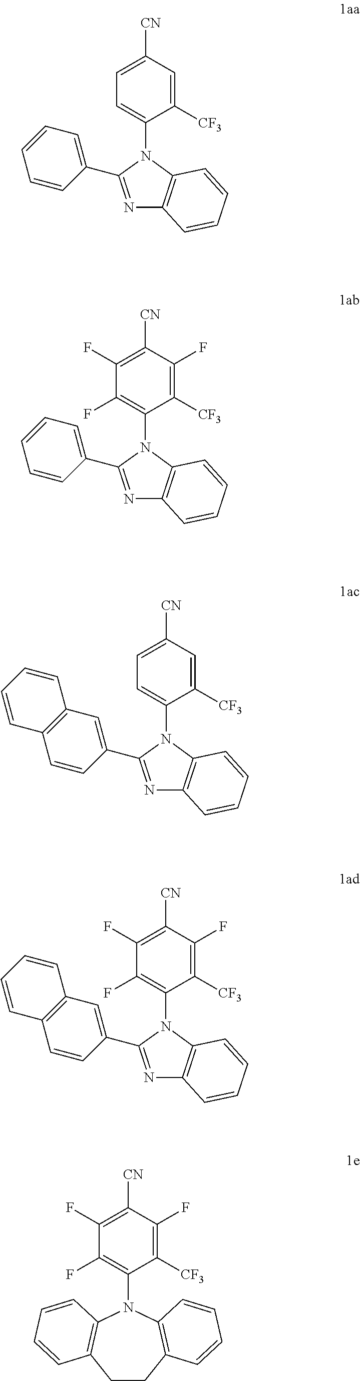

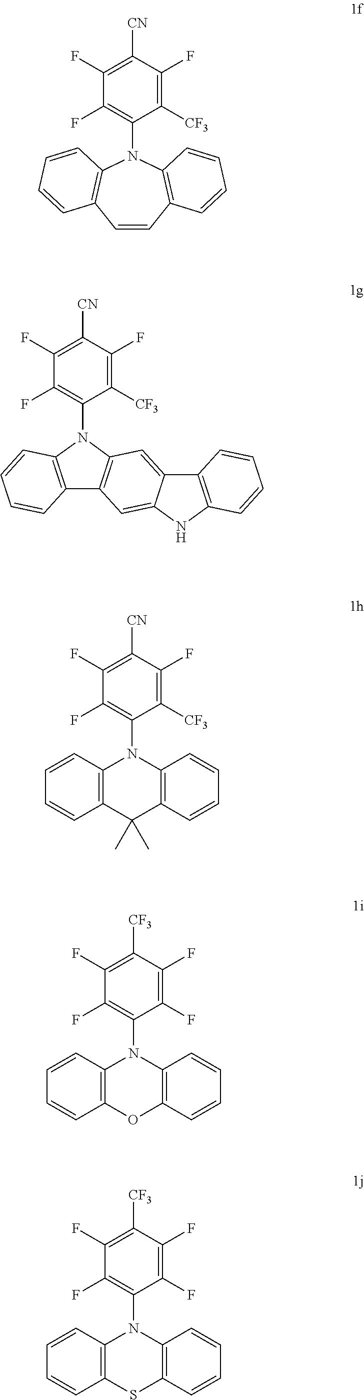

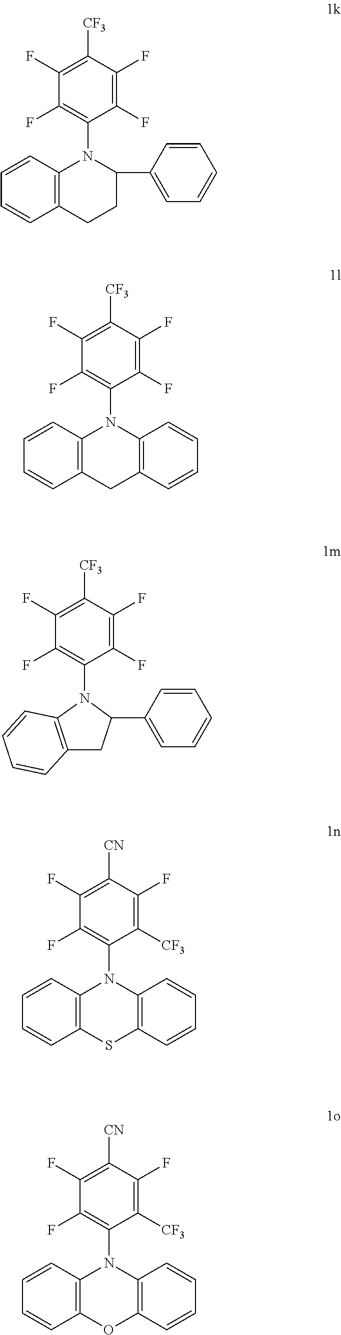

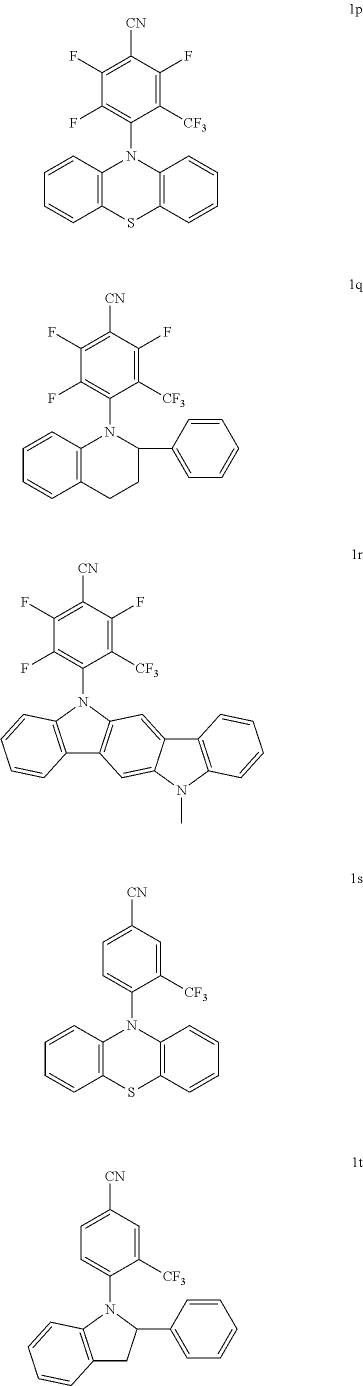

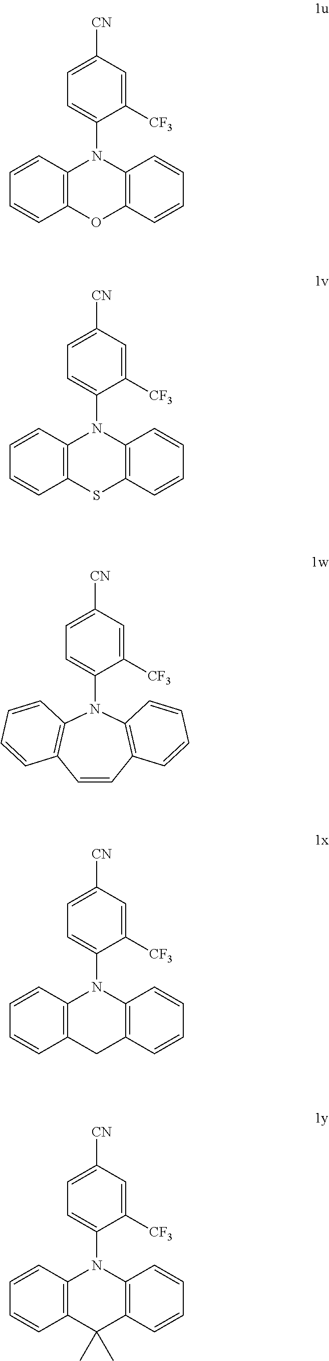

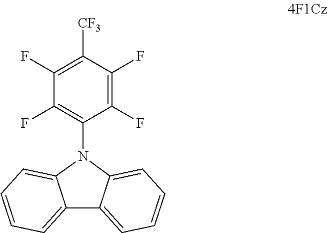

In one embodiment, the compound of the general formula (I) is a compound of the general formula (III):

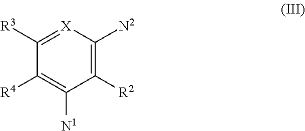

In the compound of the general formula (III), X denotes C—CF3, C—SCN, C—CCl3, C—Cl, C—F, C—CN or N; N1 and N2 each independently denote a nitrogen-containing heteroaryl radical bonded via a nitrogen atom, or a radical of the formula —N(Ar)2, —N(HetAr)(Ar), —N(HetAr)2, where each Ar is independently an aryl radical and each HetAr is independently a heteroaryl radical; and R2, R3 and R4 each independently denote F, Cl, Br, C1-C12 perfluoroalkyl, CF3, SCN, CN or H, with the proviso that only one substituent R2,3,4 is CN, and that, when X is C—CN, none of the substituents R2,3,4 is CN.

In various embodiments of the compounds of the formula (III), the same selection criteria as defined above for compounds of the formula (Ia) are applicable to the nitrogen-containing heteroaryl radical (N1,2).

In various embodiments of the compounds of the formula (III), in addition, the same selection criteria as defined above for compounds of the formula (Ia) are applicable to aryl radicals in the radicals of the formulae —N(Ar)2 and —N(HetAr)(Ar) and to heteroaryl radicals in the radicals of the formulae —N(HetAr)(Ar) and —N(HetAr)2.

In various embodiments, the following definitions are applicable to the substituents of the compound of the formula (III):

The substituent X denotes C—CF3, C—Cl, or N; the substituents N1 and N2 each independently denote a substituted or unsubstituted quinoline, benzimidazole, phenoxazine, benzomorpholine, benzothiomorpholine, dibenzazepine, carbazole or 9H-acridine radical, each of which is bonded to the aromatic system of the formula (III) via the nitrogen atom, such as a 2-phenyl-1,2,3,4-tetrahydroquinoline, 2-phenylbenzimidazole, phenoxazine, 3-phenylbenzomorpholine, 3-phenylbenzothiomorpholine, dibenzazepine, 10,11-dihydrobenzazepine, 2,3-indolocarbazole, 2,3-(1-methylindolo)carbazole, 9H-acridine or 9,9-dimethylacridine radical, each of which is bonded to the aromatic system of the formula (III) via the nitrogen atom in the main carbon ring; the substituent R2 denotes CN, CF3, F or Cl; and the substituents R3 and R4 denote H, CN, CF3, F or Cl.

Specific embodiments of the compounds of the general formula (III) are the compounds 2a-2t and 3F2Cz:

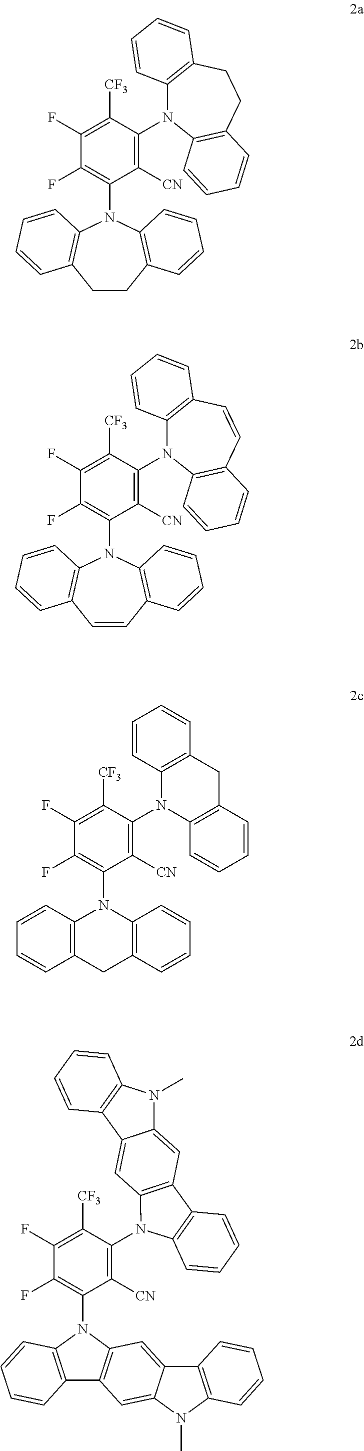

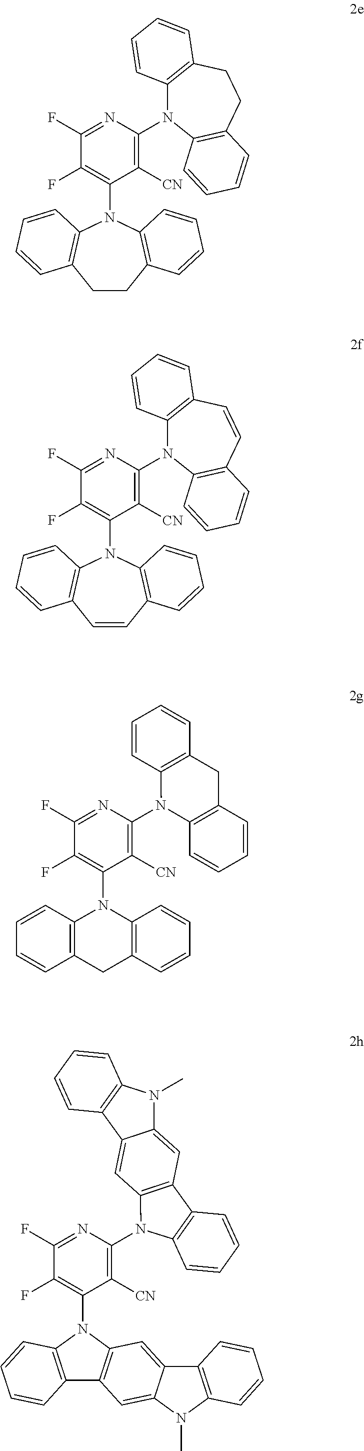

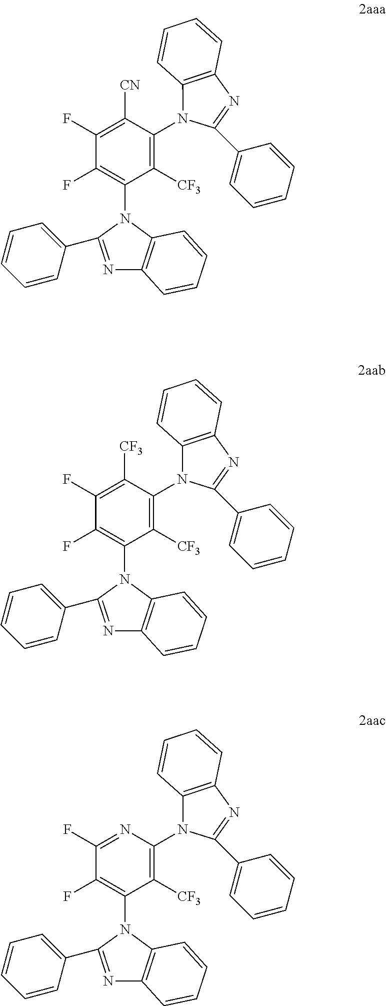

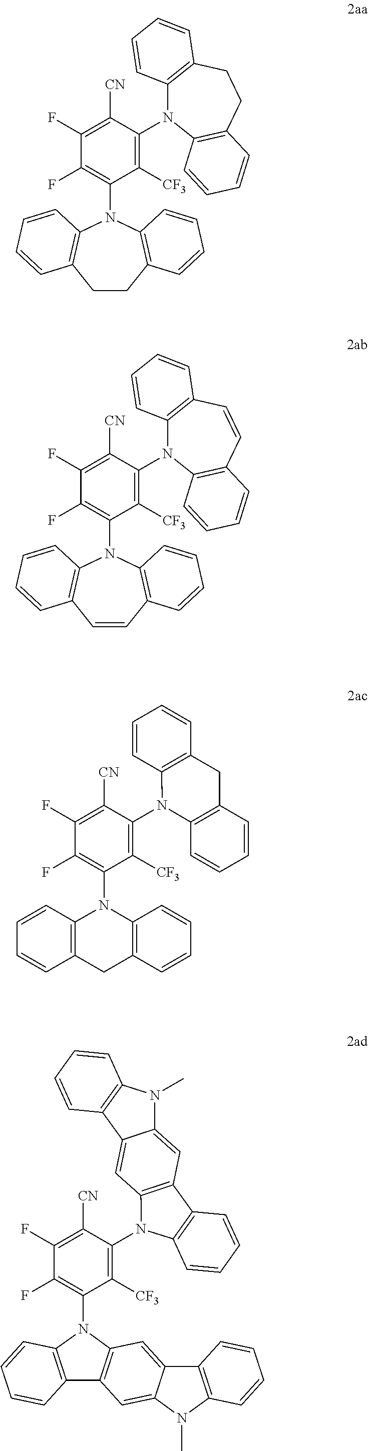

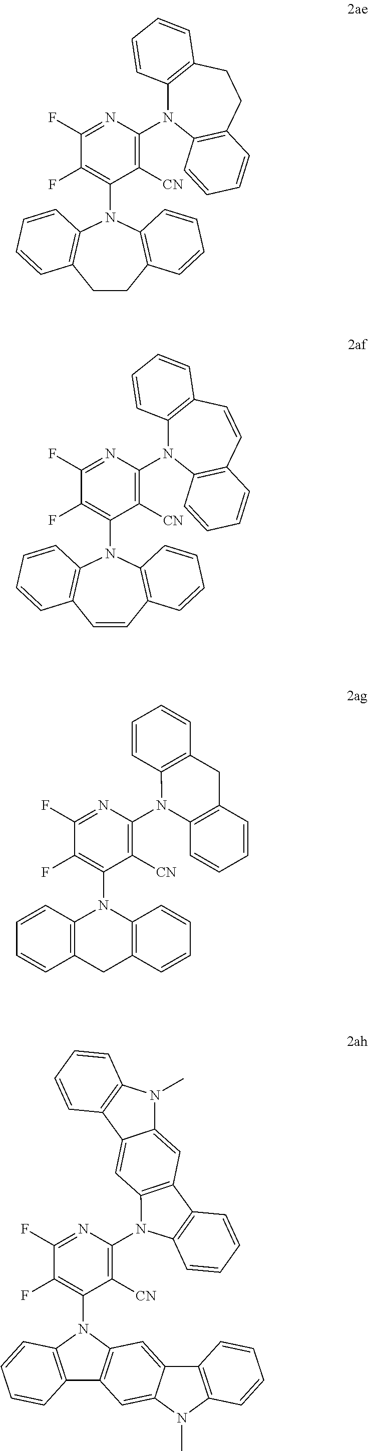

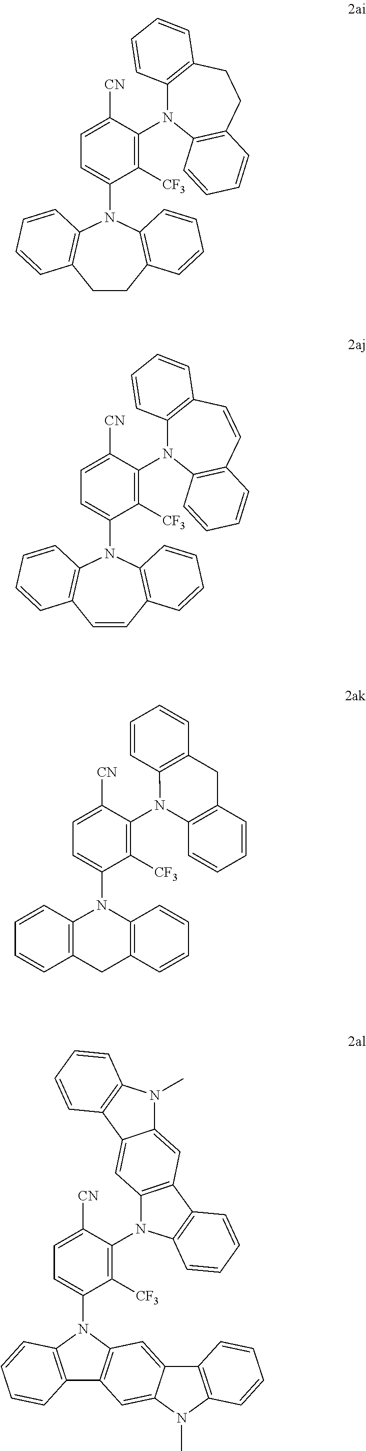

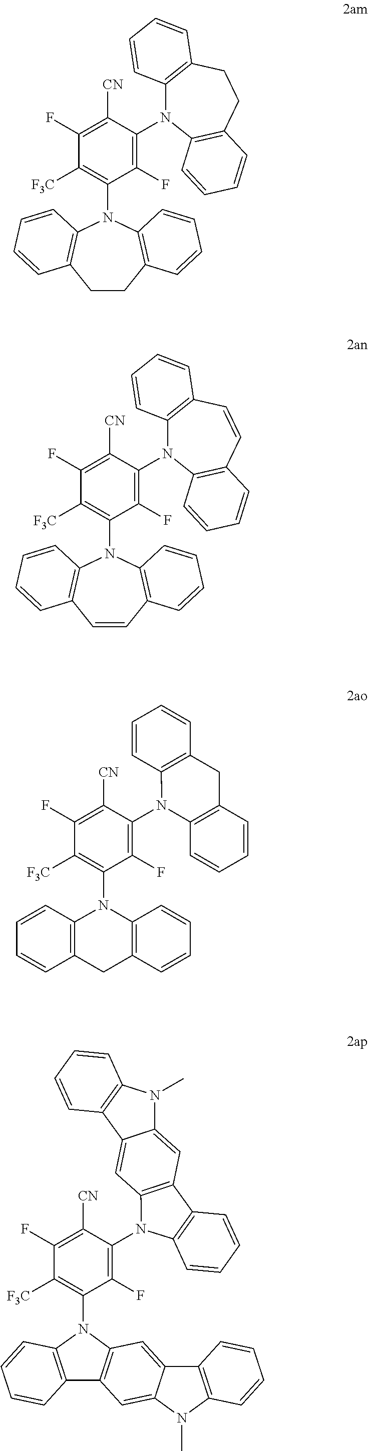

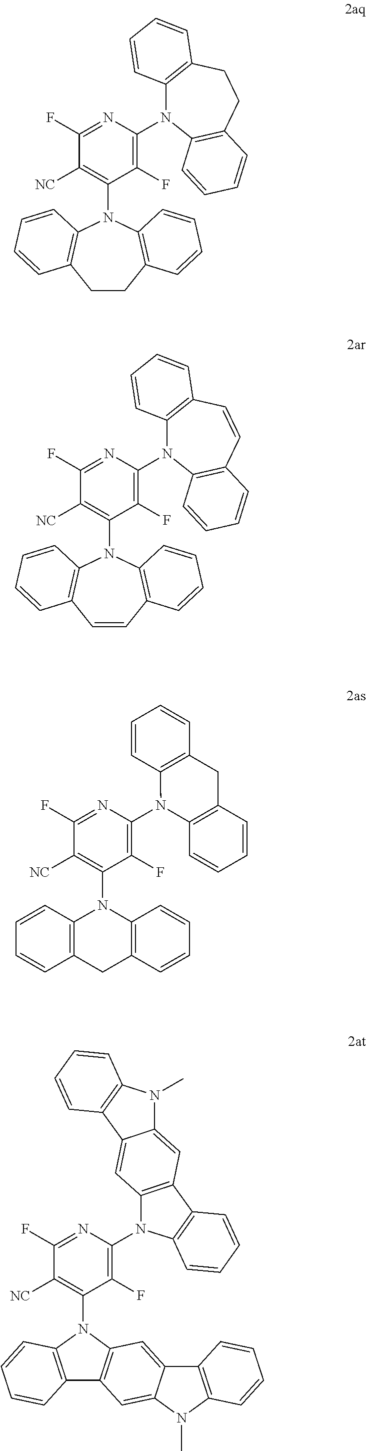

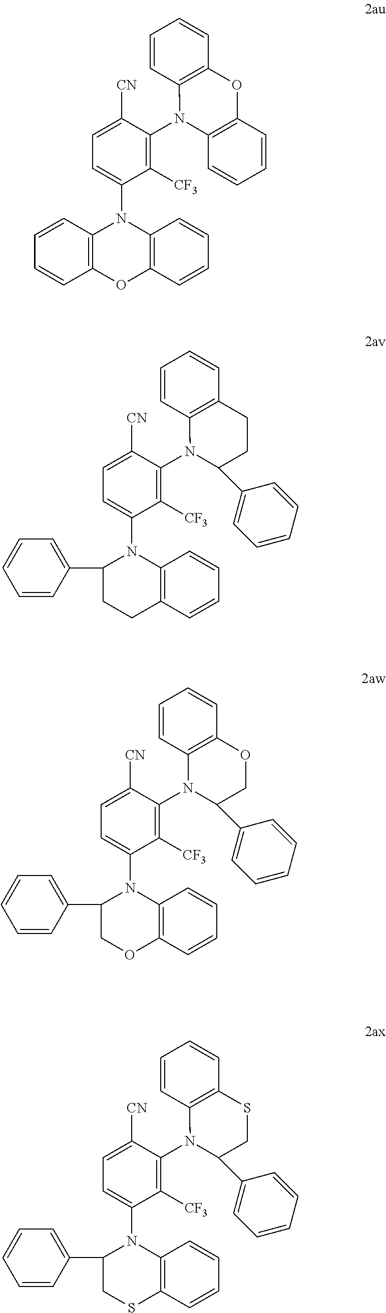

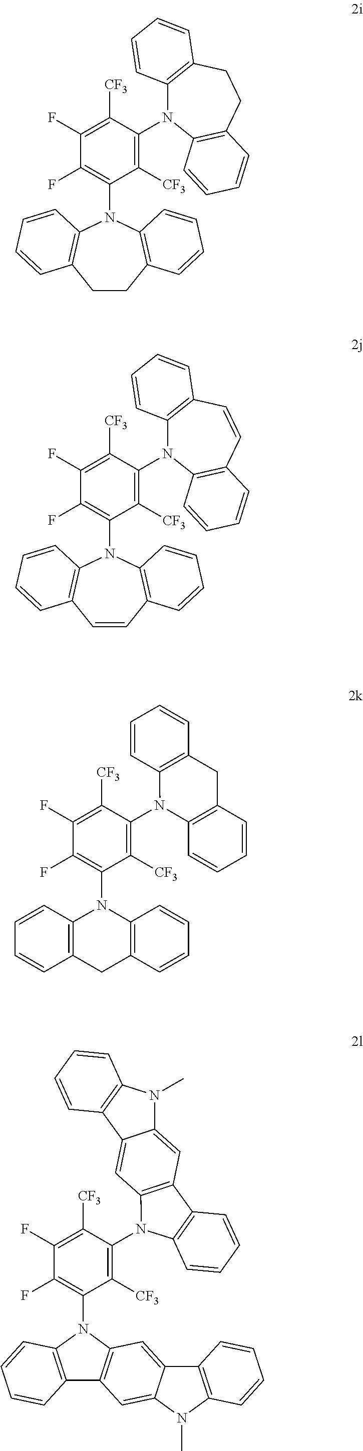

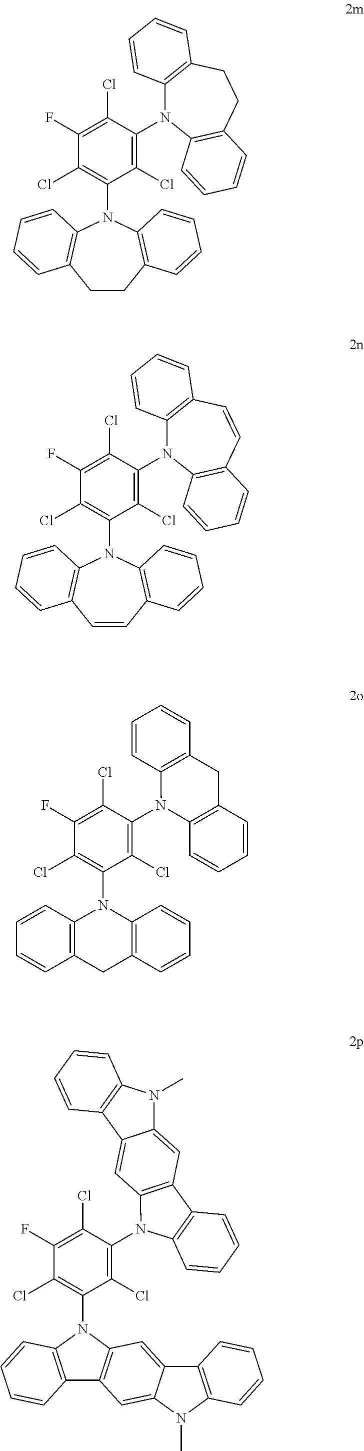

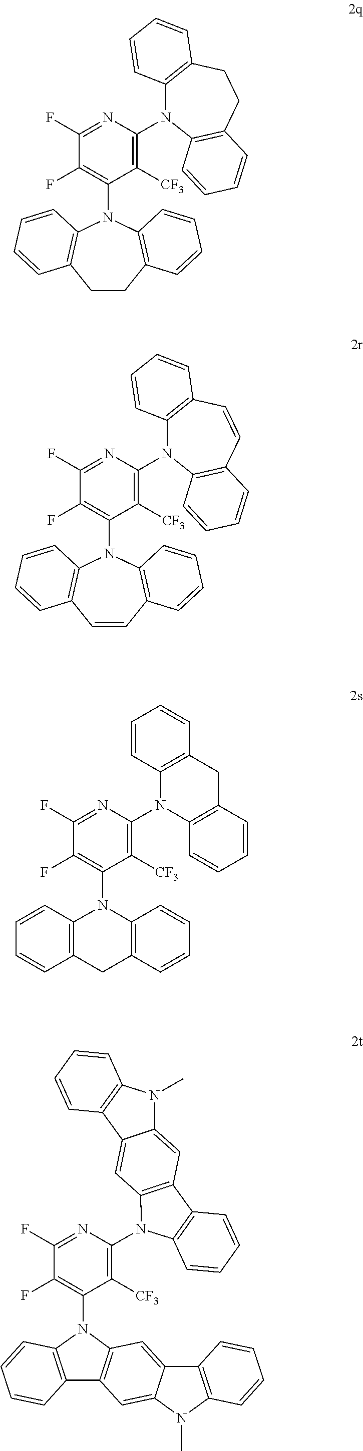

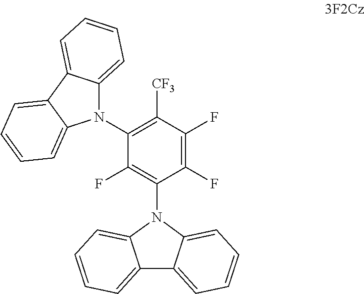

In a further embodiment, the compound of the general formula (I) is a compound of the general formula (IV):

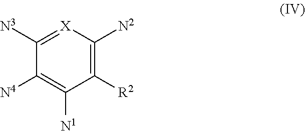

In the compound of the general formula (IV), X denotes C—CF3, C—Cl3, C—Cl, C—F, C—SCN, C—CN or N; R2 denotes F, Cl, Br, CF3, SCN, CN or H, with the proviso that R2 is not CN when X is C—CN; and N1, N2, N3 and N4 each independently denote a nitrogen-containing heteroaryl radical bonded via a nitrogen atom, or a radical of the formula —N(Ar)2, —N(HetAr)(Ar), —N(HetAr)2, where each Ar is independently an aryl radical and each HetAr is independently a heteroaryl radical.

In various embodiments of the compounds of the formula (IV), the same selection criteria as defined above for compounds of the formula (Ia) are applicable to the nitrogen-containing heteroaryl radical (N1-4).

In various embodiments of the compounds of the formula (IV), in addition, the same selection criteria as defined above for compounds of the formula (Ia) are applicable to aryl radicals in the radicals of the formulae —N(Ar)2 and —N(HetAr)(Ar) and to heteroaryl radicals in the radicals of the formulae —N(HetAr)(Ar) and —N(HetAr)2.

In various embodiments, the following definitions are applicable to the substituents of the compound of the formula (IV):

The substituent X denotes C—CN, C—CF3, C—Cl or N; the substituents N1-4 each independently denote a substituted or unsubstituted quinoline, benzomorpholine, benzothiomorpholine, indole, benzimidazole, phenoxazine, phenothiazine, dibenzazepine, carbazole or 9H-acridine radical, each of which is bonded to the aromatic system of the formula (IV) via the nitrogen atom, such as a 2-phenylindole, 2-phenylbenzimidazole, 1,4-dihydro-2-phenylquinoline, 3-phenyl-1,4-benzoxazine, 3-phenyl-1,4-benzothiazine, phenoxazine, phenothiazine, dibenzazepine, 10,11-dihydrobenzazepine, 2,3-indolocarbazole, 2,3-(1-methylindolo)carbazole, or 9,9-dimethylacridine radical, each of which is bonded to the aromatic system of the formula (IV) via the nitrogen atom in the main carbon ring; and the substituent R2 denotes CF3, SCN, F or CN.

Specific embodiments of the compounds of the general formula (IV) are the compounds 3a-31f, 4aaa-4aae and 1F4Cz:

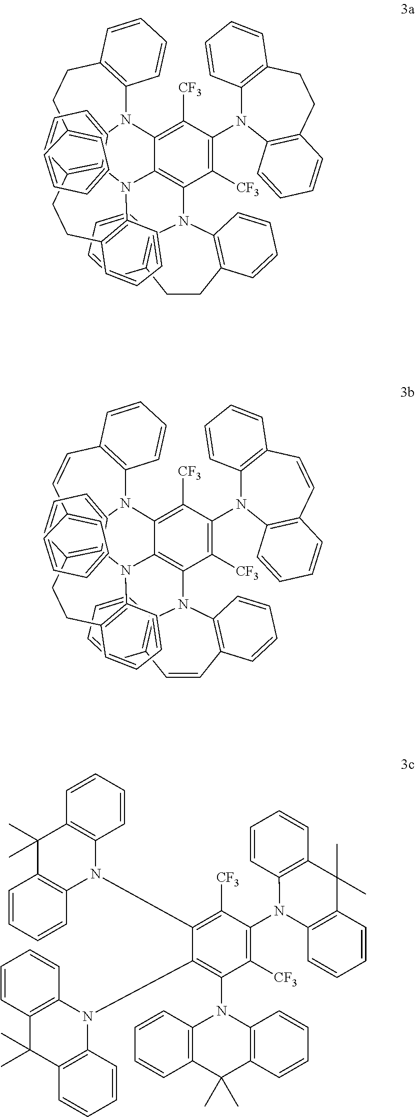

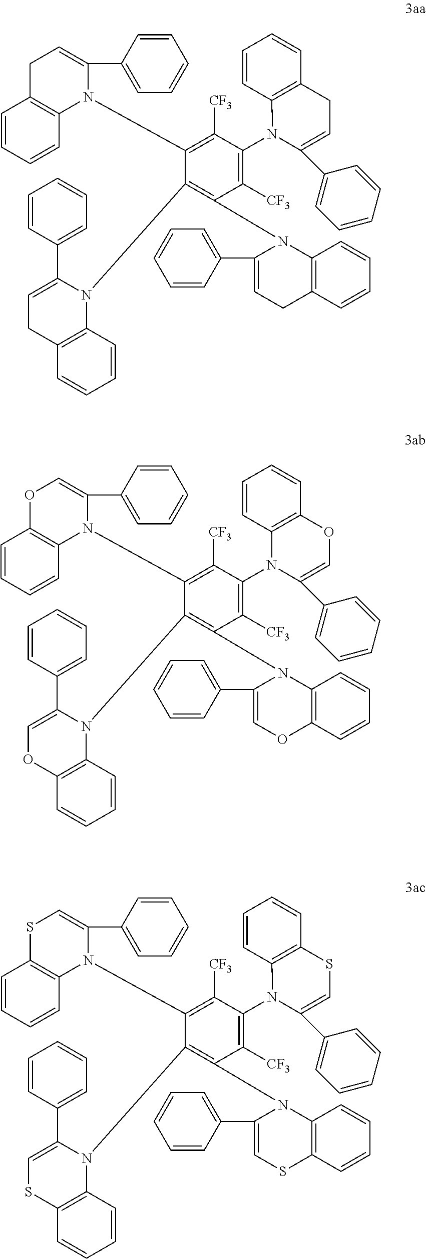

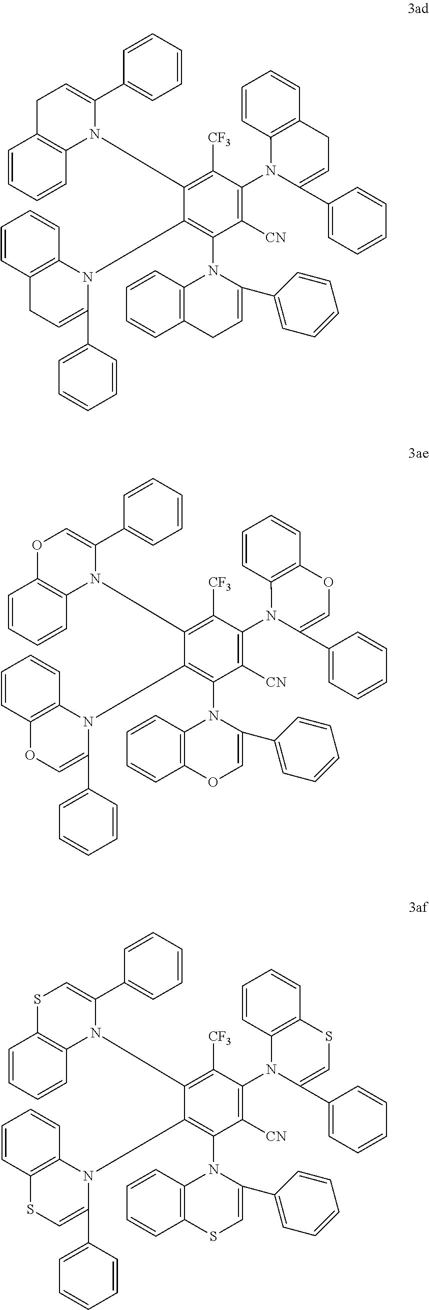

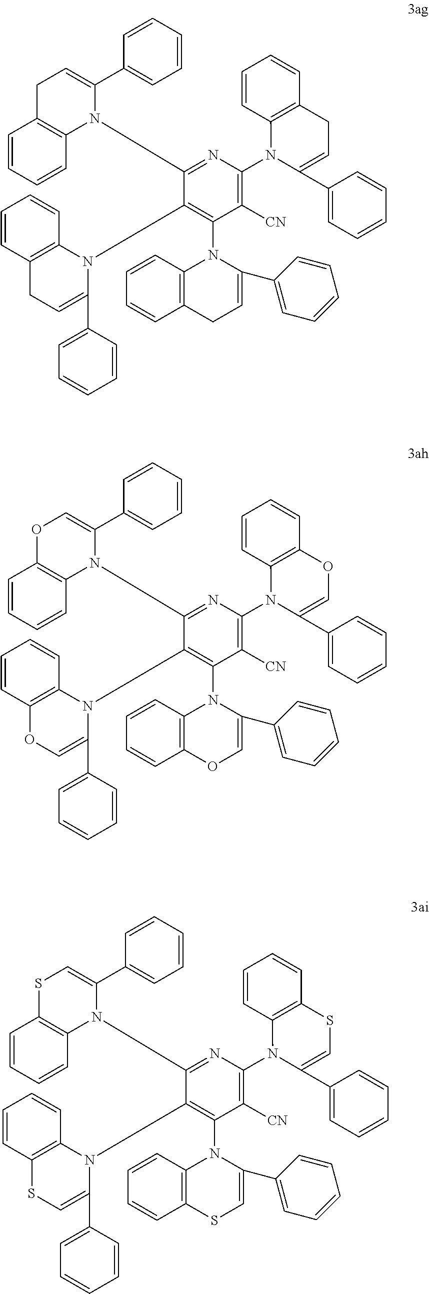

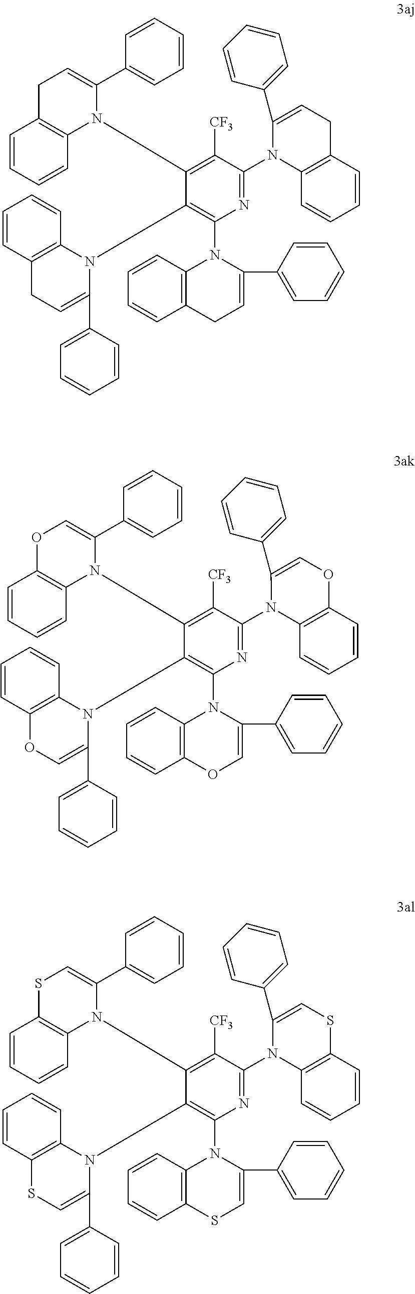

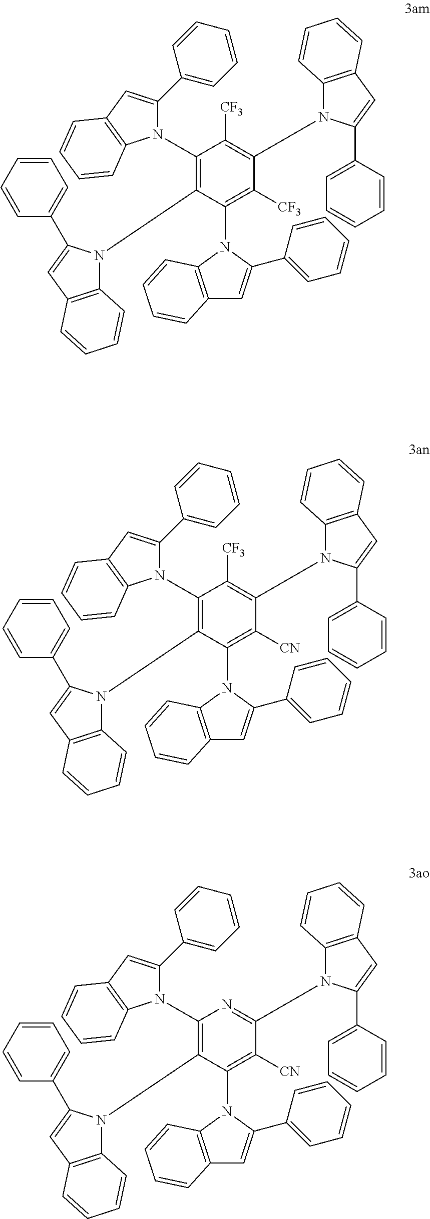

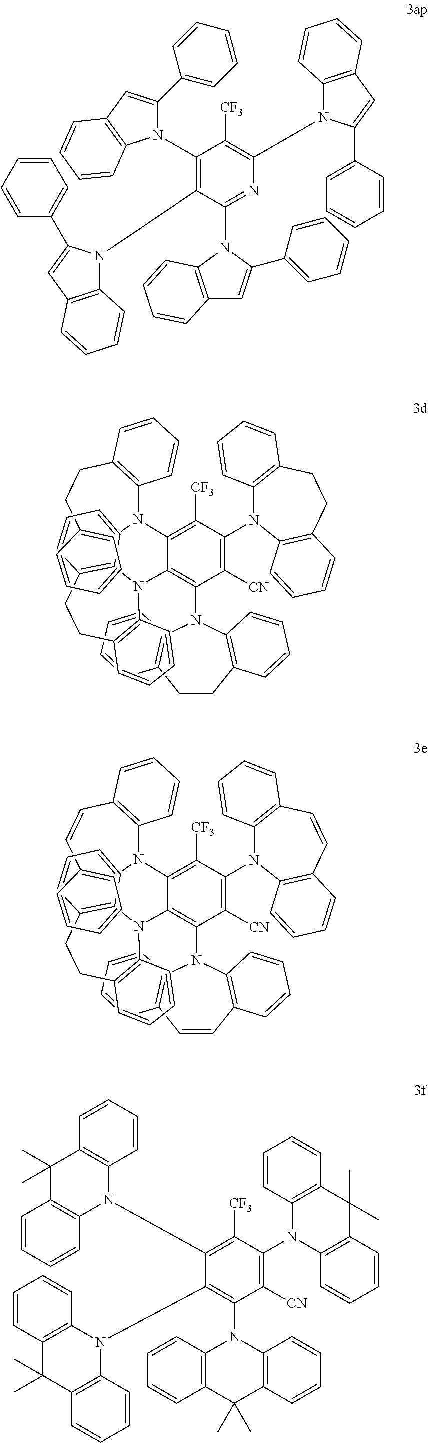

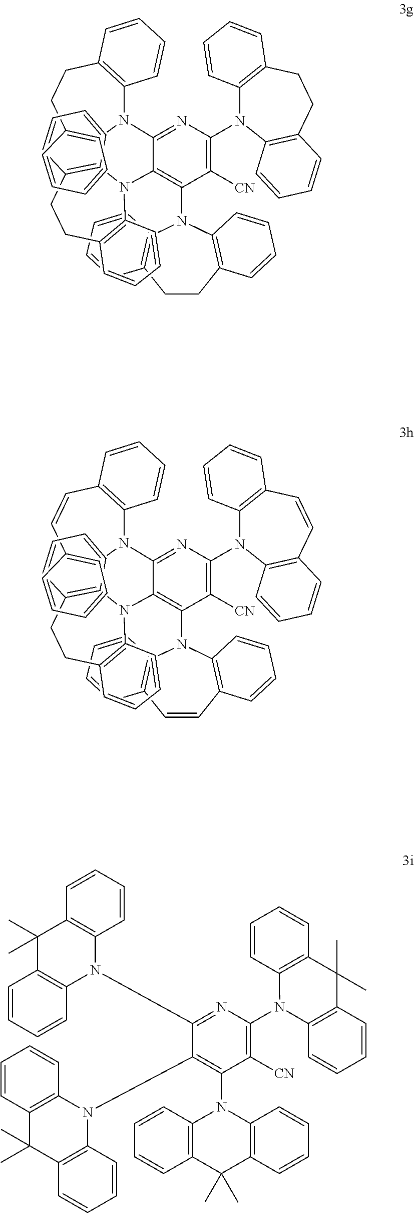

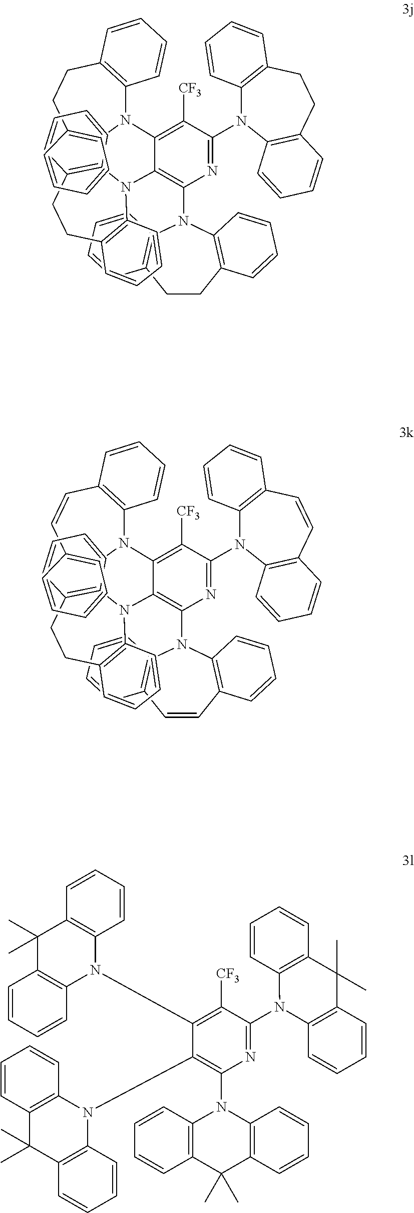

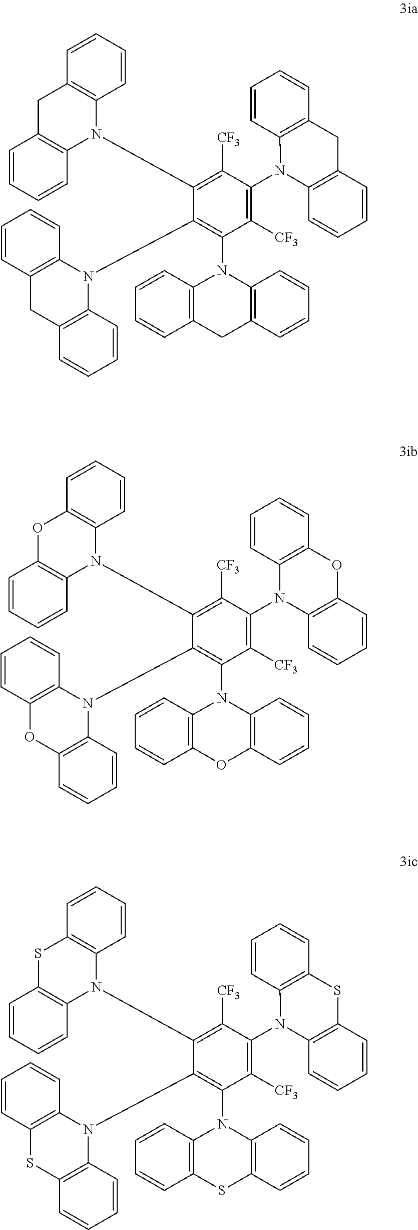

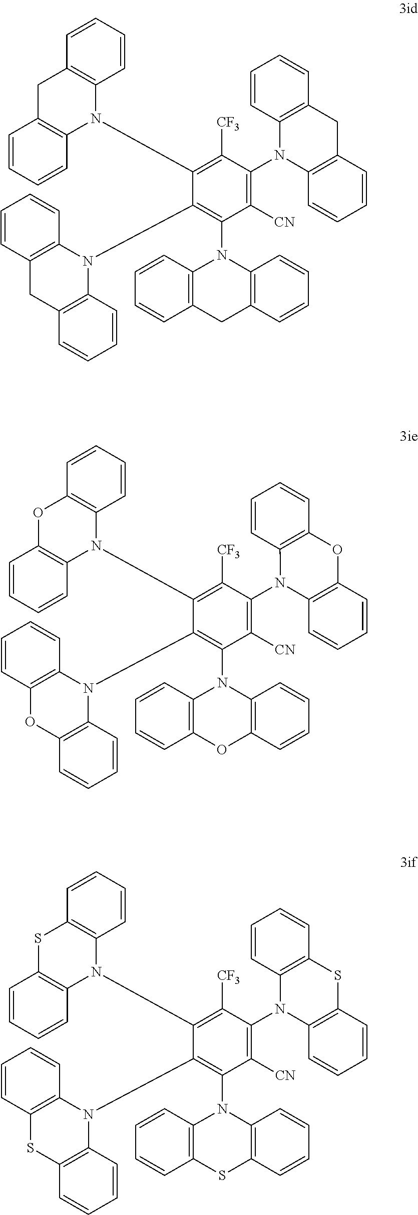

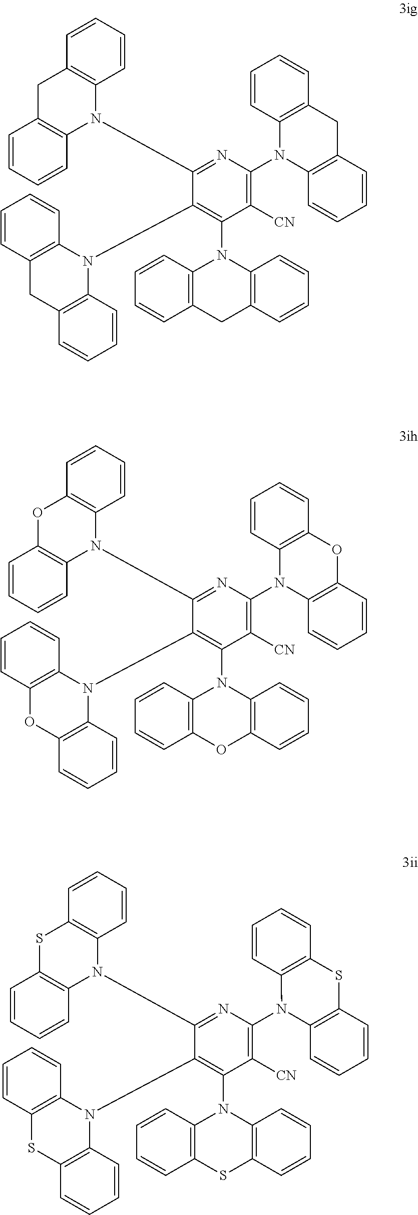

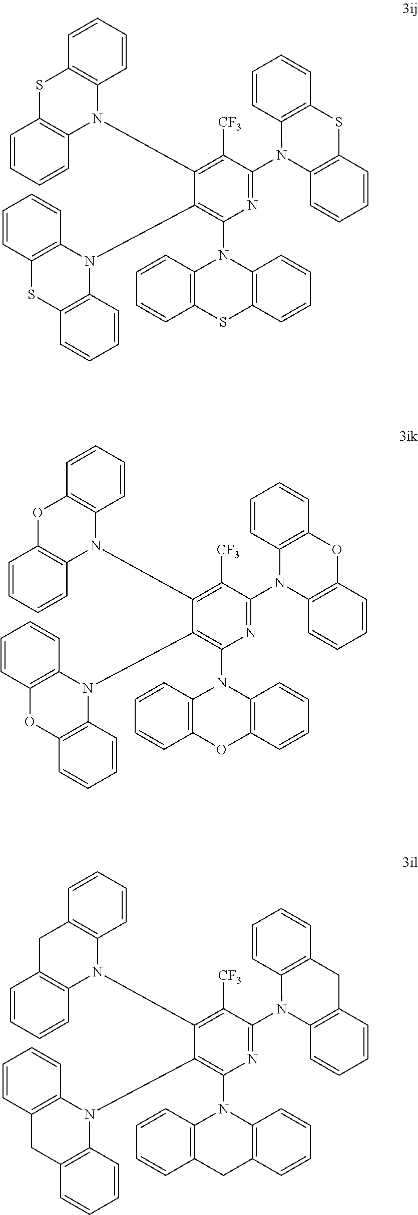

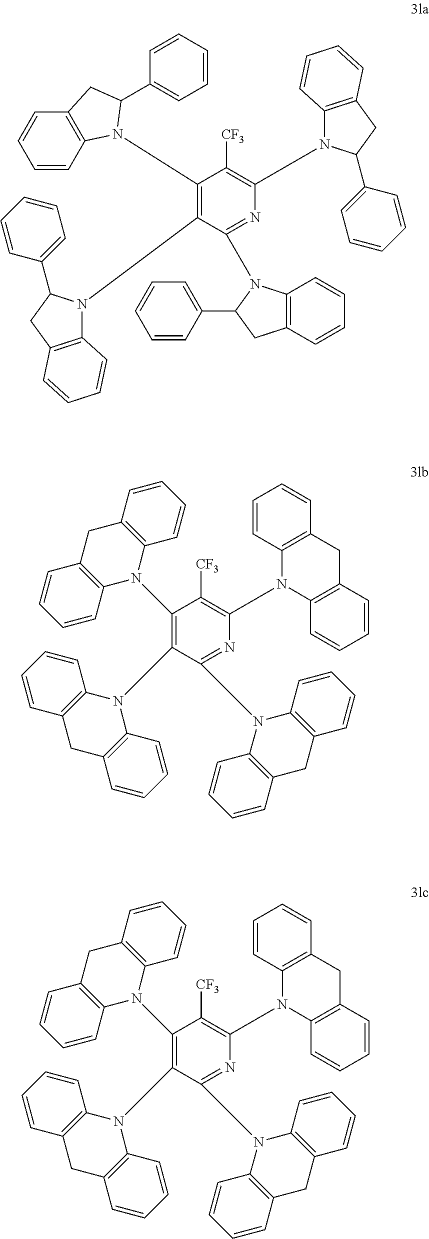

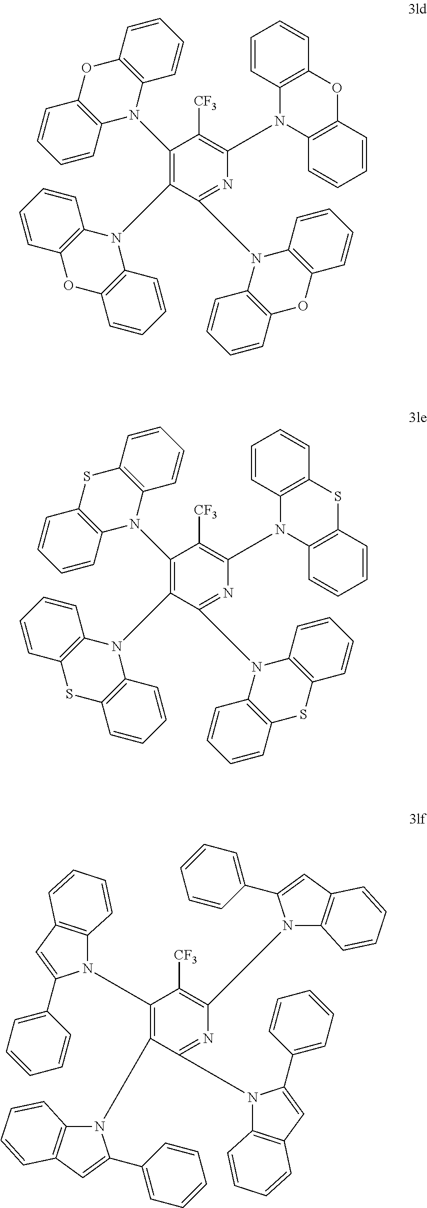

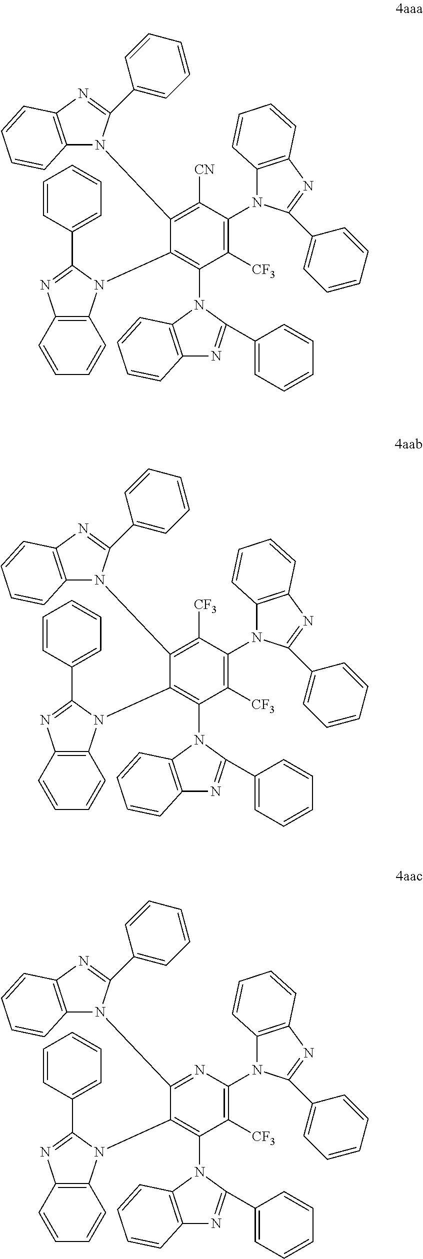

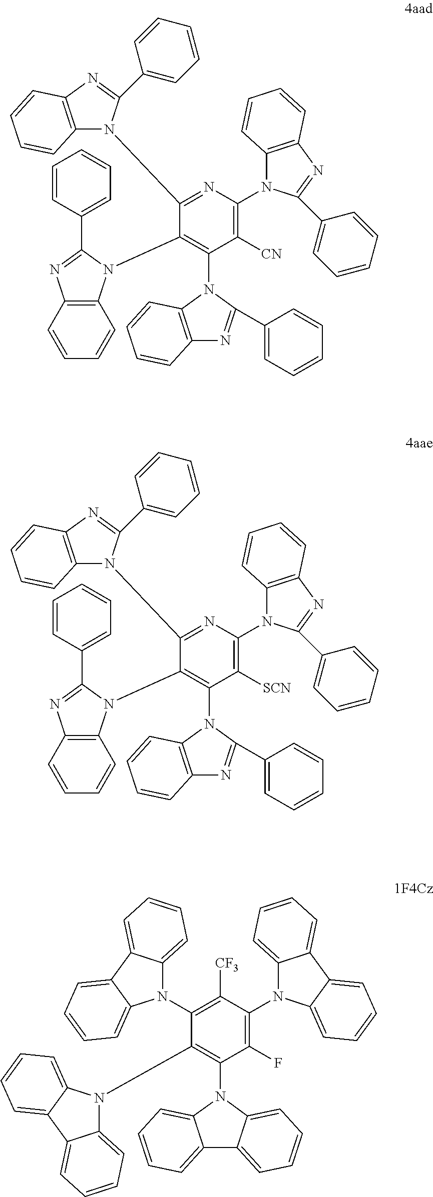

In various embodiments, the compounds of the formula (IV) are restricted to those compounds in which, when X is C—CN and N1, N2, N3 and N4 are each a carbazole bonded via the nitrogen atom, R2 is not F.

In further embodiments, the compound of the general formula (I) is a compound of the general formula (Va)-(Vd):

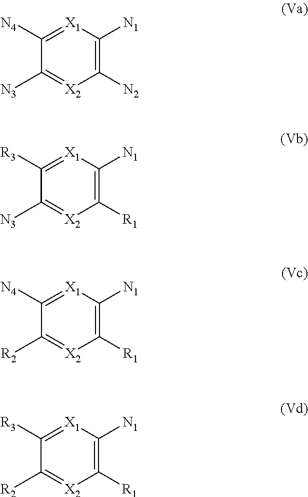

In the compound of the general formula (Va), X1 and X2 each independently denote C—CF3, C—SCN, C—Cl, C—F, C—CN or N; and N1, N2, N3 and N4 each independently denote a nitrogen-containing heteroaryl radical bonded via a nitrogen atom, or a radical of the formula —N(Ar)2, —N(HetAr)(Ar), —N(HetAr)2, where each Ar is independently an aryl radical and each HetAr is independently a heteroaryl radical. This is subject to the proviso that X2 is not C—CN when X1 is C—CN, C—F or C—Cl.

In the compound of the general formula (Vb), X1 and X2 each independently denote C—CF3, C—SCN, C—Cl, C—F, C—CN or N; N1 and N3 each independently denote a nitrogen-containing heteroaryl radical bonded via a nitrogen atom, or a radical of the formula —N(Ar)2, —N(HetAr)(Ar), —N(HetAr)2, where each Ar is independently an aryl radical and each HetAr is independently a heteroaryl radical; and R1 and R3 each independently denote H or F.

In the compound of the general formula (Vc), X1 and X2 each independently denote C—CF3, C—SCN, C—Cl, C—F, C—CN or N; N1 and N4 each independently denote a nitrogen-containing heteroaryl radical bonded via a nitrogen atom, or a radical of the formula —N(Ar)2, —N(HetAr)(Ar), —N(HetAr)2, where each Ar is independently an aryl radical and each HetAr is independently a heteroaryl radical; and R1 and R2 each independently denote H or F.

In the compound of the general formula (Vd), X1 and X2 each independently denote C—CF3, C—SCN, C—Cl, C—F, C—CN or N; N1 denotes a nitrogen-containing heteroaryl radical bonded via a nitrogen atom, or a radical of the formula —N(Ar)2, —N(HetAr)(Ar), —N(HetAr)2, where each Ar is independently an aryl radical and each HetAr is independently a heteroaryl radical; and R1, R2 and R3 each independently denote H or F.

In various embodiments of the compounds of the formula (Va)-(Vd), the same selection criteria as defined above for compounds of the formula (Ia) are applicable to the nitrogen-containing heteroaryl radical (N1-4).

In various embodiments of the compounds of the formula (Va)-(Vd), in addition, the same selection criteria as defined above for compounds of the formula (Ia) are applicable to aryl radicals in the radicals of the formulae —N(Ar)2 and —N(HetAr)(Ar) and to heteroaryl radicals in the radicals of the formulae —N(HetAr)(Ar) and —N(HetAr)2.

In various embodiments, the following definitions are applicable to the substituents of the compound of the formula (Va):

The substituents X1 and X2 each independently denote C—CF3, C—CN or N, where X1 and X2 are not simultaneously C—CN; and the substituents N1-4 each independently denote a substituted or unsubstituted benzimidazole, dibenzazepine, carbazole or 9H-acridine radical, each of which is bonded to the aromatic system of the formula (Va) via the nitrogen atom, such as a 2-phenylbenzimidazole, dibenzazepine, 10,11-dihydrobenzazepine or 9,9-dimethylacridine radical, each of which is bonded to the aromatic system of the formula (Va) via the nitrogen atom.

Specific embodiments of the compounds of the general formula (Va) are the compounds VIIa-VIIs:

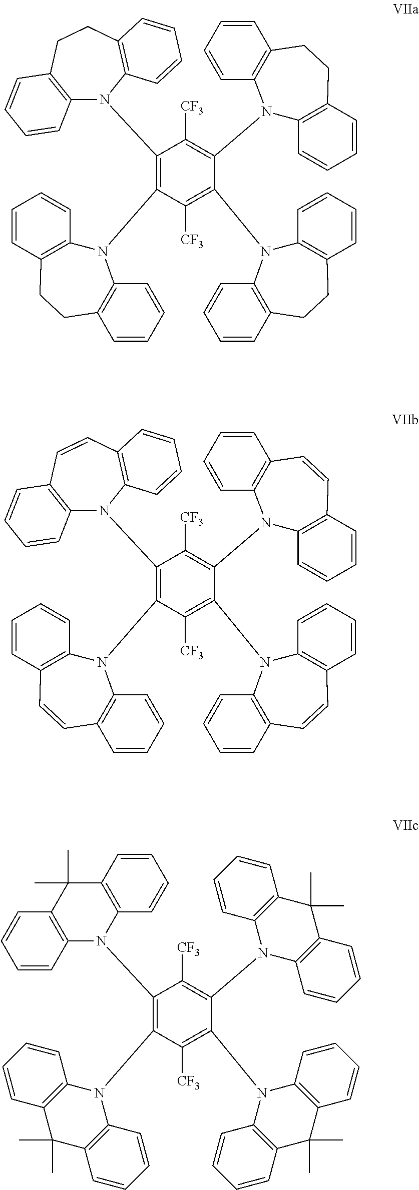

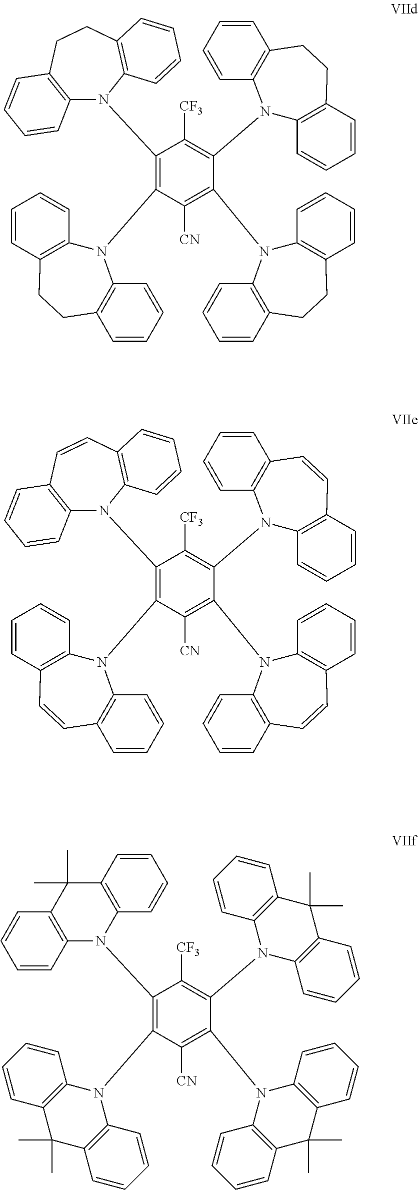

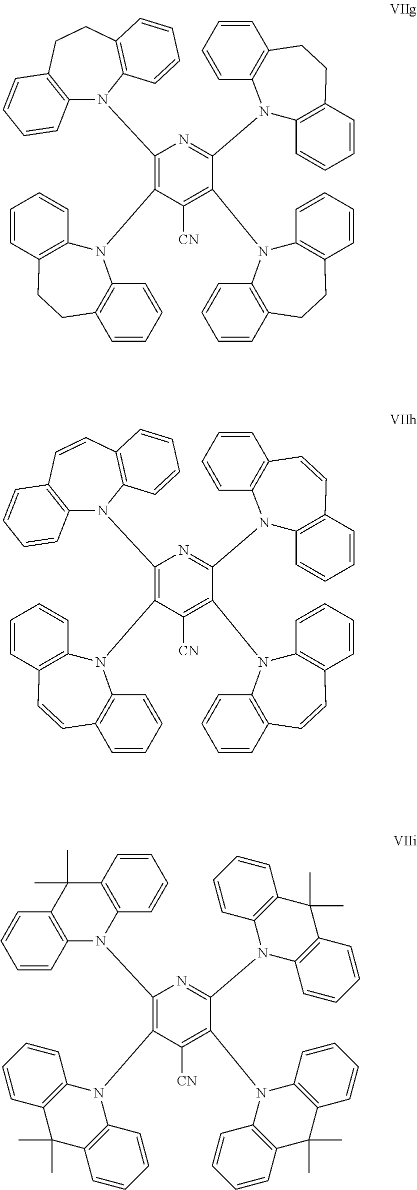

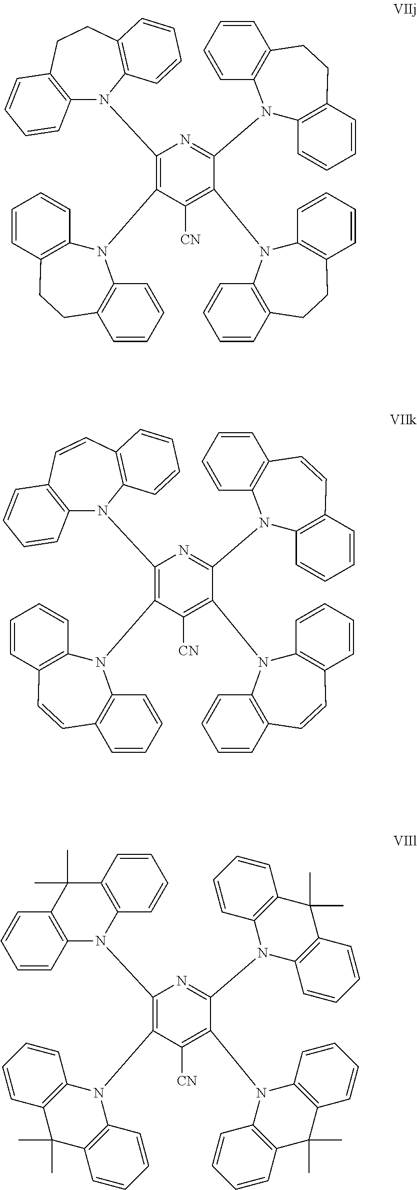

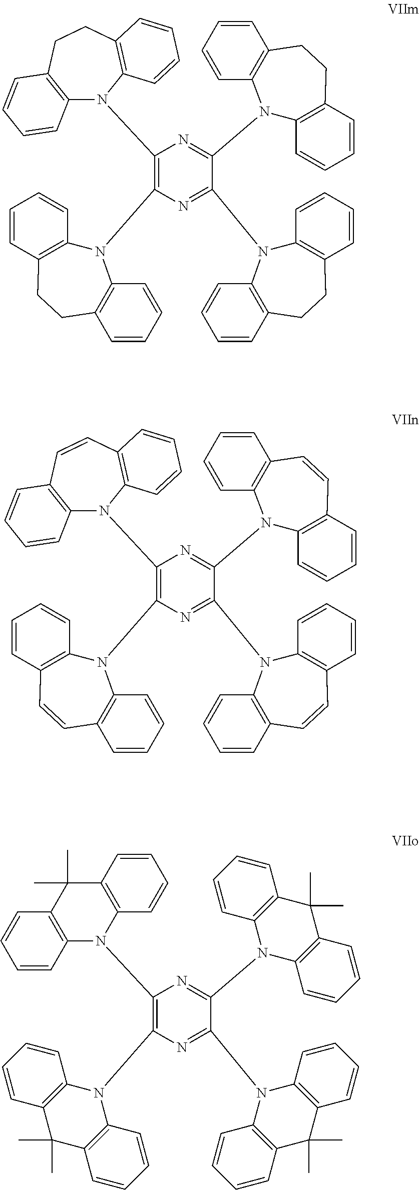

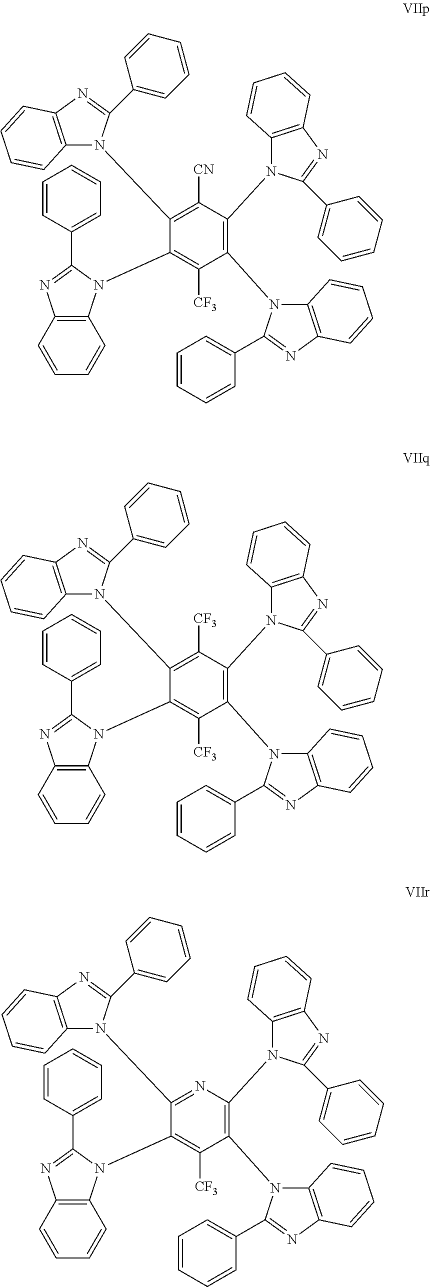

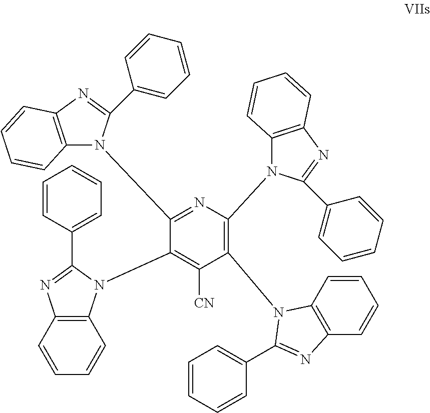

In various embodiments, the following definitions are applicable to the substituents of the compound of the formula (Vb):

The substituents X1 and X2 each independently denote C—CF3, C—CN or N, where X1 and X2 are not simultaneously C—CN; the substituents N1 and N3 each independently denote a substituted or unsubstituted benzimidazole, dibenzazepine, carbazole or 9H-acridine radical, each of which is bonded to the aromatic system of the formula (Vb) via the nitrogen atom, such as a dibenzazepine, 10,11-dihydrobenzazepine, carbazole, 9H-acridine, 9,9-dimethylacridine or 2-phenylbenzimidazole radical, each of which is bonded to the aromatic system of the formula (Vb) via the nitrogen atom; and the substituents R1 and R3 each independently denote H or F.

Specific embodiments of the compounds of the general formula (Vb) are the compounds VIIt-VHz and Xa-Xe:

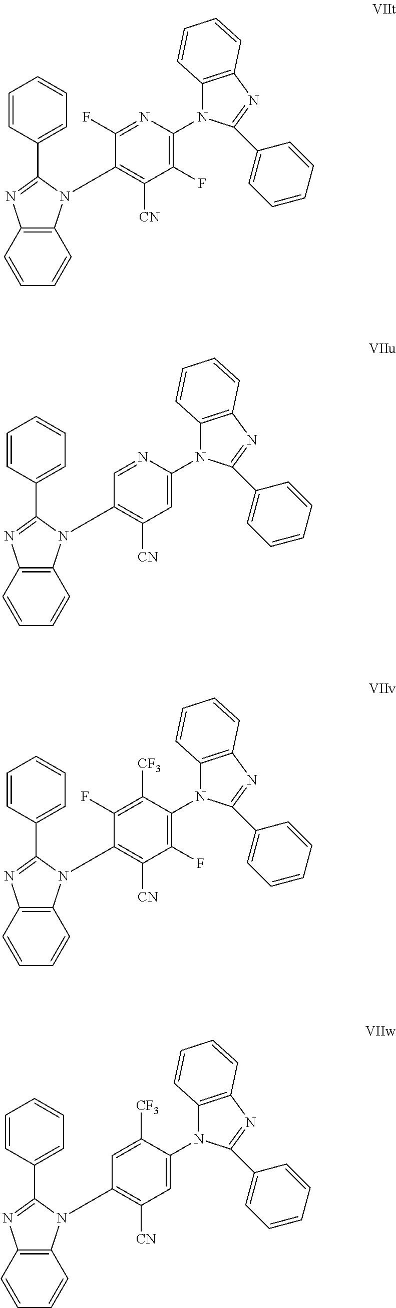

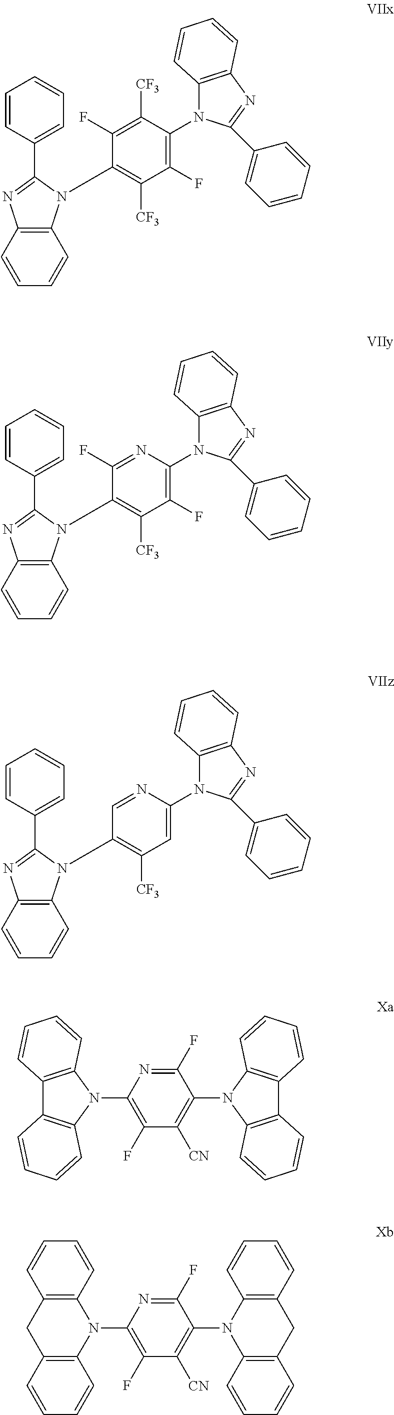

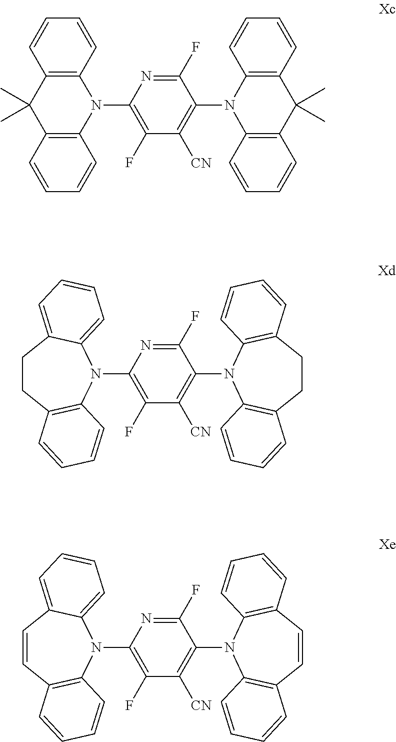

In various embodiments, the following definitions are applicable to the substituents of the compound of the formula (Vc):

The substituents X1 and X2 each independently denote C—CF3, C—CN or N, where X1 and X2 are not simultaneously C—CN; the substituents N1 and N4 each independently denote a substituted or unsubstituted benzimidazole, dibenzazepine, carbazole or 9H-acridine radical, each of which is bonded to the aromatic system of the formula (Vc) via the nitrogen atom, such as a 2-phenylbenzimidazole, carbazole, dibenzazepine, 10,11-dihydrobenzazepine, 9H-acridine or 9,9-dimethylacridine radical, each of which is bonded to the aromatic system of the formula (Vc) via the nitrogen atom; and the substituents R1 and R2 are each independently H or F.

A specific embodiment of the compounds of the general formula (Vc) is the compound VIIzz and Xf-Xj:

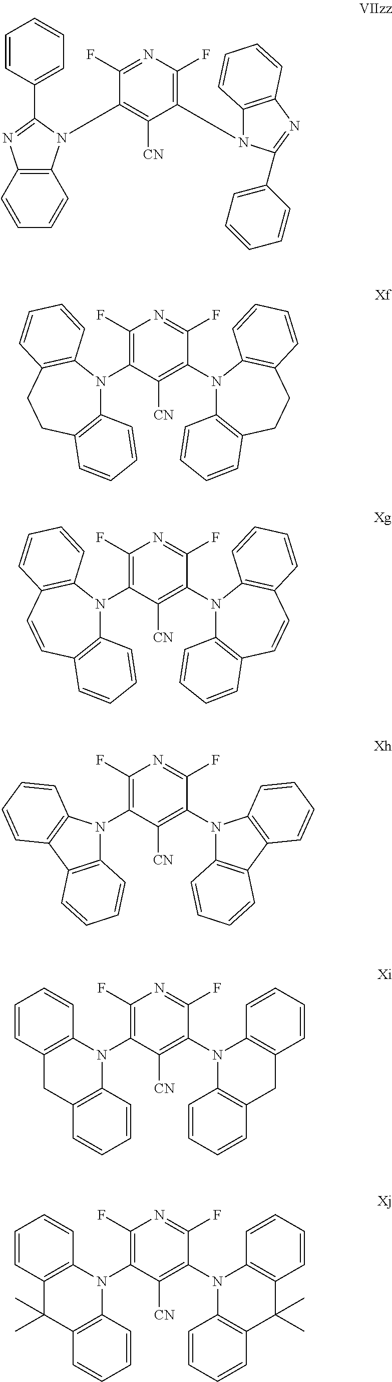

In various embodiments, the following definitions are applicable to the substituents of the compound of the formula (Vd):

The substituent X1 denotes C—CF3, C—CN or N; the substituent X2 denotes N; the substituent N1 denotes a substituted or unsubstituted dibenzazepine, carbazole or 9H-acridine radical, each of which is bonded to the aromatic system of the formula (Vd) via the nitrogen atom, such as a carbazole, 9H-acridine, 9,9-dimethylacridine, dibenzazepine or 10,11-dihydrobenzazepine radical, each of which is bonded to the aromatic system of the formula (Vd) via the nitrogen atom; and the substituents R1, R2 and R3 each independently denote H or F.

Specific embodiments of the compounds of the general formula (Vd) are the compounds VIIaa-VIIae:

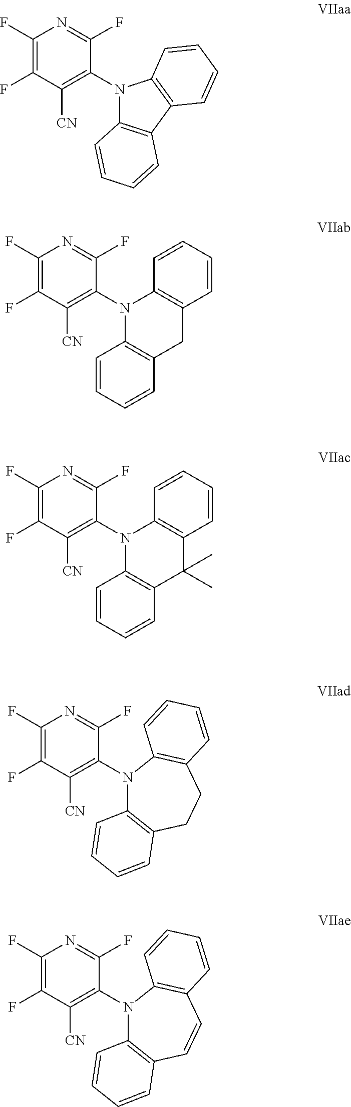

In various embodiments, the compound of the general formula (I) is a compound of the general formula (VIa) or (VIb):

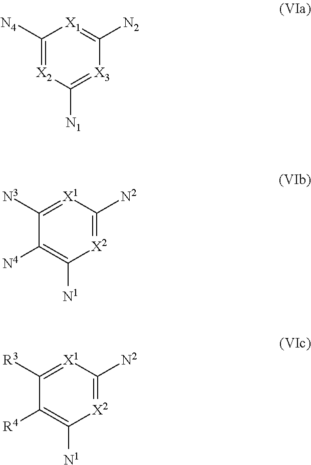

In the compounds of the general formula (VIa), X1, X2 and X3 each independently denote C—CF3, C—CCl3, C—Cl, C—F, C—CN or N; and N1, N2 and N4 each independently denote a nitrogen-containing heteroaryl radical bonded via a nitrogen atom, or a radical of the formula —N(Ar)2, —N(HetAr)(Ar), —N(HetAr)2, where each Ar is independently an aryl radical and each HetAr is independently a heteroaryl radical. This is subject to the proviso that X1, X2 and X3 are not all simultaneously C—CN, C—Cl, C—F or N, and, when X1 and X2 are each C—CN, X3 is C—CF3 or N.

In the compounds of the general formula (VIb), X1 and X2 each denote N; and N1, N2, N3 and N4 each independently denote a nitrogen-containing heteroaryl radical bonded via a nitrogen atom, or a radical of the formula —N(Ar)2, —N(HetAr)(Ar), —N(HetAr)2, where each Ar is independently an aryl radical and each HetAr is independently a heteroaryl radical.

In the compounds of the general formula (VIc), X1 denotes C—CN or N; X2 denotes N; N1 and N2 each independently denote a nitrogen-containing heteroaryl radical bonded via a nitrogen atom, or a radical of the formula —N(Ar)2, —N(HetAr)(Ar), —N(HetAr)2, where each Ar is independently an aryl radical and each HetAr is independently a heteroaryl radical; and R3 and R4 each independently denote H or F.

In various embodiments of the compounds of the formulae (VIa)-(VIc), the same selection criteria as defined above for compounds of the formula (Ia) are applicable to the nitrogen-containing heteroaryl radical (N1-4).

In various embodiments of the compounds of the formulae (VIa)-(VIc), in addition, the same selection criteria as defined above for compounds of the formula (Ia) are applicable to aryl radicals in the radicals of the formulae —N(Ar)2 and —N(HetAr)(Ar) and to heteroaryl radicals in the radicals of the formulae —N(HetAr)(Ar) and —N(HetAr)2.

In various embodiments, the following definitions are applicable to the substituents of the compounds of the formula (VIa):

The substituents X1-3 each independently denote C—CF3, C—CN, C—Cl, C—F or N; and the substituents N1,2,4 each independently denote a substituted or unsubstituted dibenzazepine, carbazole or 9H-acridine radical, each of which is bonded to the aromatic system of the formula (VI) via the nitrogen atom, such as a dibenzazepine, 10,11-dihydrobenzazepine, or 9,9-dimethylacridine radical, each of which is bonded to the aromatic system of the formula (VI) via the nitrogen atom.

In various embodiments, in the compounds of the formula (VIa), the substituents X1-3 each independently denote C—CF3, C—CN, C—Cl or C—F, and the substituents N1,2,4 each independently denote a dibenzazepine, 10,11-dihydrobenzazepine, or 9,9-dimethylacridine radical, each of which is bonded to the aromatic system of the formula (VI) via the nitrogen atom.

In further embodiments, in the compounds of the formula (VIa), the substituent X1 denotes N; the substituents X2 and X3 each independently denote C—CF3, C—F or C—Cl; and the substituents N1,2,4 each independently denote a dibenzazepine, 10,11-dihydrobenzazepine, or 9,9-dimethylacridine radical, each of which is bonded to the aromatic system of the formula (VIa) via the nitrogen atom.

In further embodiments, in the compounds of the formula (VIa), the substituent X3 denotes N; the substituents X1 and X2 each independently denote C—CF3, C—F or C—Cl; and the substituents N1,2,4 each independently denote a dibenzazepine or 10,11-dihydrobenzazepine radical, each of which is bonded to the aromatic system of the formula (VIa) via the nitrogen atom.

In still further embodiments, in the compounds of the formula (VIa), the substituents X2 and X3 each denote N; the substituent X1 denotes C—CF3, C—F or C—Cl; and the substituents N1,2,4 each independently denote a dibenzazepine or 10,11-dihydrobenzazepine radical, each of which is bonded to the aromatic system of the formula (VIa) via the nitrogen atom.

In still further embodiments, in the compounds of the formula (VIa), the substituents X1 and X2 each denote N; the substituent X3 denotes C—CF3, C—F, C—Cl or C—CN; and the substituents N1,2,4 each independently denote a dibenzazepine or 10,11-dihydrobenzazepine radical, each of which is bonded to the aromatic system of the formula (VIa) via the nitrogen atom.

Specific embodiments of the compounds of the general formula (VIa) are the compounds 5a-5l, 6a-6i and 2F3Cz:

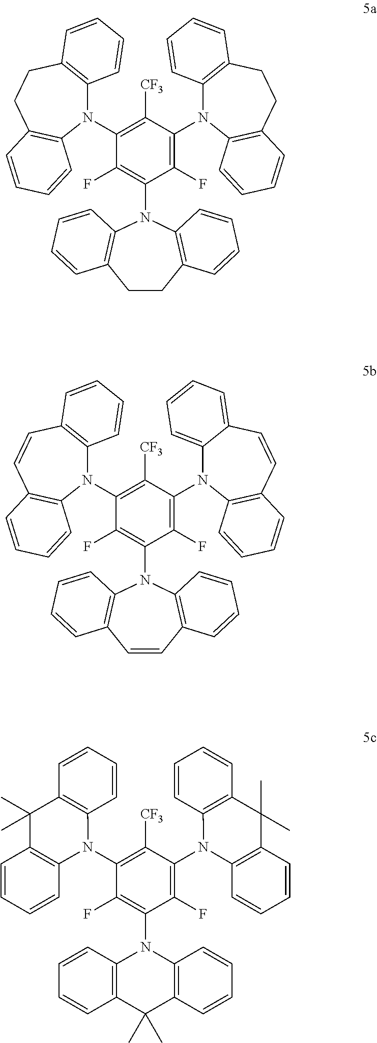

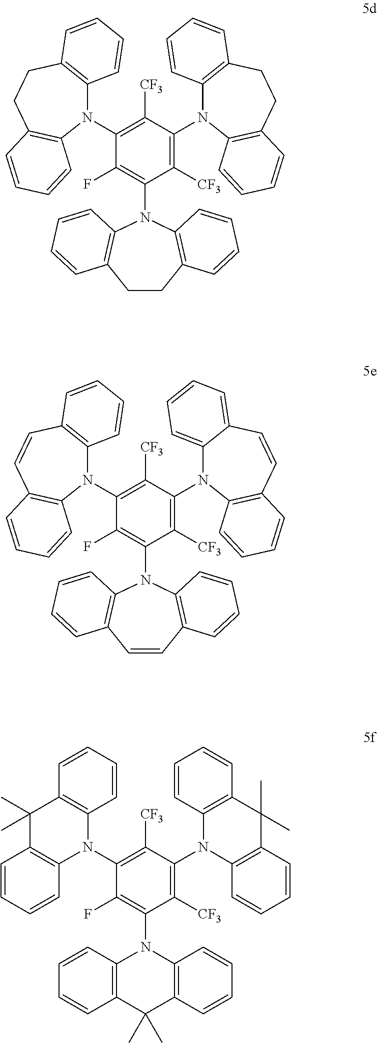

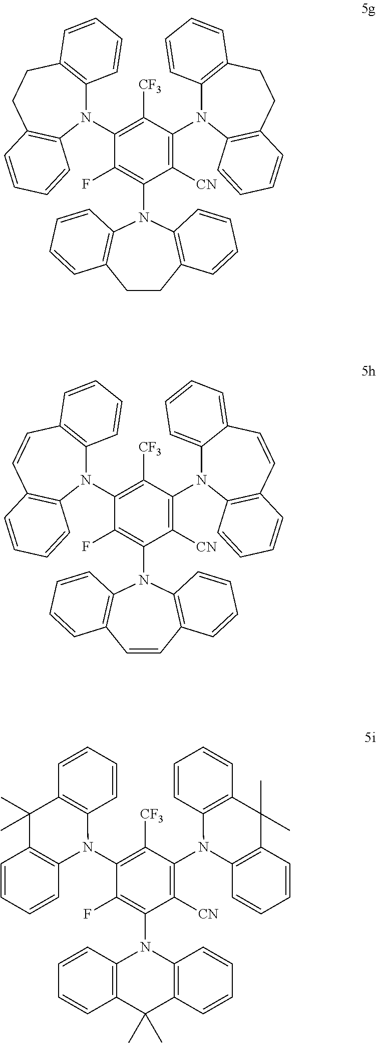

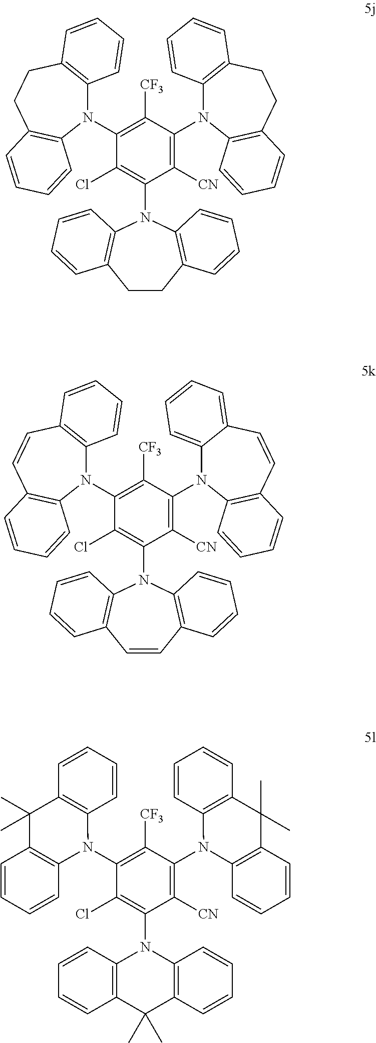

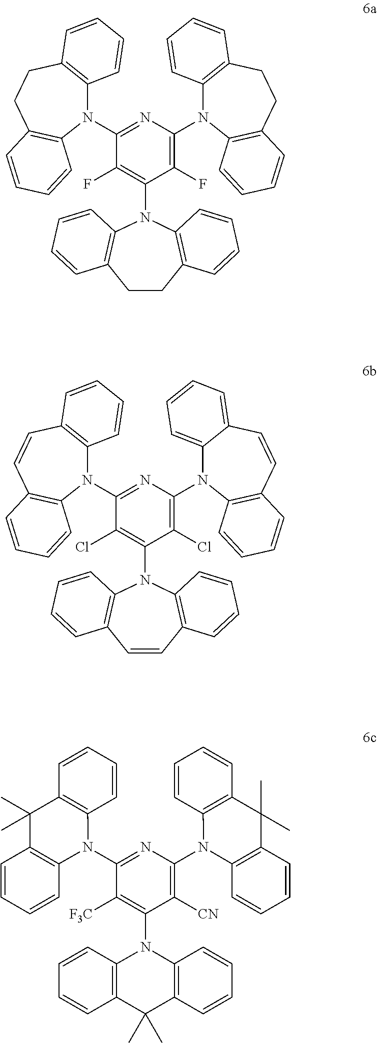

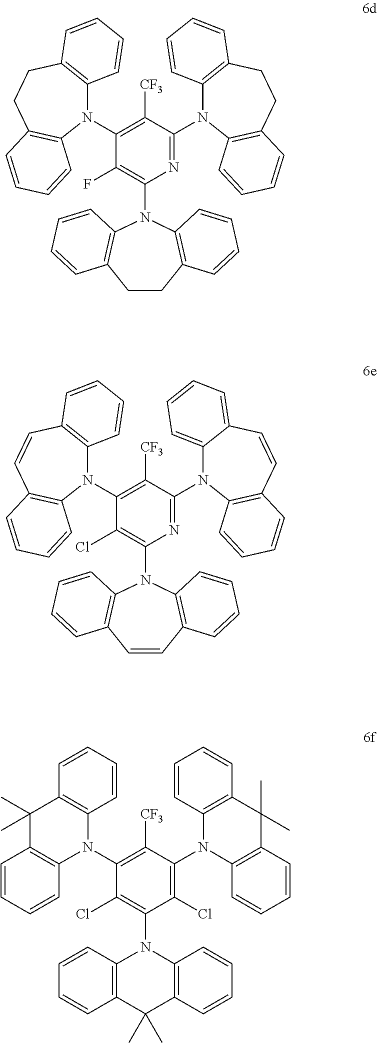

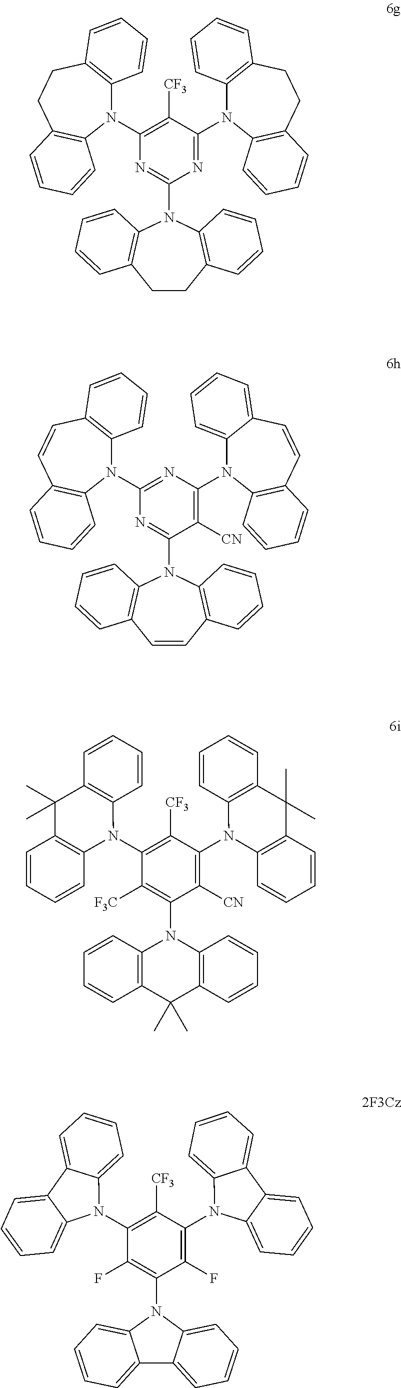

In various embodiments, the following definitions are applicable to the substituents of the compounds of the formula (VIb):

The substituents X1 and X2 each denote N; and the substituents N1-4 each independently denote a substituted or unsubstituted indole, benzimidazole, dibenzazepine, carbazole or 9H-acridine radical, each of which is bonded to the aromatic system of the formula (VIb) via the nitrogen atom, such as a 2-phenylindole or 2-phenylbenzimidazole radical, each of which is bonded to the aromatic system of the formula (VIb) via the nitrogen atom.

Specific embodiments of the compounds of the general formula (VIb) are the compounds 3aq and 4aaf:

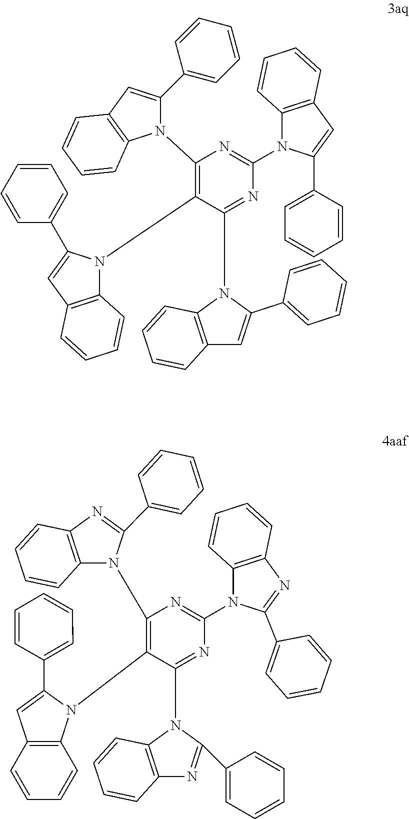

Another non-limiting embodiment is additionally directed to the use of at least one compound of the formula (I) in an optoelectronic component.

In various embodiments, the following definitions are applicable to the substituents of the compounds of the formula (VIc):

The substituent X1 denotes C—CN or N; the substituent X2 denotes N; the substituents N1 and N2 each independently denote a substituted or unsubstituted indole, benzimidazole, dibenzazepine, carbazole or 9H-acridine radical, each of which is bonded to the aromatic system of the formula (VIc) via the nitrogen atom, such as a 2-phenylindole or 2-phenylbenzimidazole radical, each of which is bonded to the aromatic system of the formula (VIc) via the nitrogen atom; and R3 and R4 each independently denote H or F.

A specific embodiment of the compounds of the general formula (VIc) is the compound 2aad:

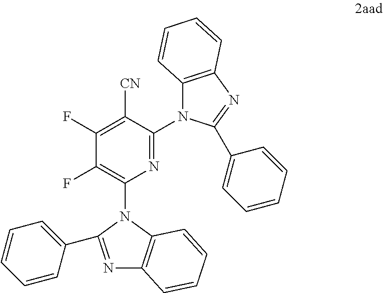

In various embodiments, the compounds of the formulae (VIa) are restricted to those compounds in which, when one of X1, X2 and X3 is C—CN and the rest of X1, X2 and X3 are each F, not all N1, N2 and N4 are simultaneously a carbazole bonded via the nitrogen atom in each case.

In various embodiments, the optoelectronic component is OSCs having a photoactive organic layer. This photoactive layer includes low molecular weight compounds, oligomers, polymers or mixtures thereof as organic coating materials. An opaque or semitransparent electrode has been applied as outer contact layer to this thin-layer component.

In a further embodiment, the optoelectronic component is disposed on a flexible substrate.

A flexible substrate is understood to mean a substrate which assures deformability as a result of external forces. This makes flexible substrates of this kind suitable for arrangement on curved surfaces as well. Flexible substrates include, for example, plastic films or metal foils, but are not limited thereto.

In various embodiments, the coating for production of an optoelectronic component is effected by means of vacuum processing of the organic compounds. In various embodiments, the compounds used for production of the optoelectronic component therefore have a low evaporation temperature, such sa<300° C., alternatively <250° C., but not lower than 150° C. In various embodiments, however, the evaporation temperature is at least 120° C. It is particularly advantageous when the organic compounds are sublimable under high vacuum.

In a further configuration, it may be the case that the coating for production of an optoelectronic component is effected by means of solution processing of the compounds described herein. The availability of commercial spray robots means that this application method can advantageously be scaled up in a simple manner to the industrial scale in roll-to-roll methods.

In various embodiments, the optoelectronic component is a solar cell of the generic type. Such an optoelectronic component typically has a layer structure wherein the respective lowermost and uppermost layers are configured as electrode and counterelectrode for formation of electrical contacts. In various embodiments, the optoelectronic component is arranged on a substrate, for example glass, plastic (PET, etc.) or a metal ribbon. At least one organic layer comprising at least one organic compound is arranged between the near-substrate electrode and the counterelectrode. Organic compounds used here may be organic low molecular weight compounds, oligomers, polymers or mixtures. In various embodiments, the organic layer is a photoactive layer. In various embodiments of the photoactive layer, can be formed, for example, in the form of a mixed layer composed of an electron donor material and an electron acceptor material. Charge carrier transport layers may be arranged adjacent to the at least one photoactive layer. According to the configuration, these can transport electrons (=negative charges) or holes (=positive charges) from or to the respective electrodes. In various embodiments, the optoelectronic component is configured as a tandem or multiple component. In this case, at least two optoelectronic components are deposited one on top of another as a layer system. In various embodiments, it is possible for there to be adjoining additional layers for coating or encapsulation of the component or further components on or beneath the base layers and outer layers configured as contacts.

In one embodiment, the organic layer takes the form of one or more thin layers of vacuum-processed low molecular weight compounds or organic polymers. The prior art discloses a multitude of optoelectronic components based on vacuum-processed low molecular weight compounds and polymers (Walzer et al., Chemical reviews 2007, 107(4), 1233-1271; Peumans et al., J. Appl. Phys. 2003, 93(7), 3693-3722).

The organic layer is deposited on a substrate using vacuum-processable compounds of the compounds described herein. In an alternative embodiment, the organic layer is deposited on a substrate by wet-chemical means using solutions.

Typical examples of optoelectronic components comprising compounds as described above are likewise provided. In embodiments of this kind, in various embodiments, is selected from the group consisting of compounds 1a-1y, 2a-2t, 3a-31f, 4aaa-4aaf, 5a-5l, 6a-6i, VIIa-VIIzz, Xa-Xj, 4F1Cz, 3F2Cz, 2F3Cz and 1F4Cz as defined above.

In various other embodiments, the optoelectronic component comprising at least one of the compounds of the formula (I) as described herein is an organic light-emitting diode (OLED).

The OLEDs are in principle formed from several layers, for example:
1. anode
2. hole conductor layer
3. light-emitting layer
4. blocking layer for holes/excitons
5. electron conductor layer
6. cathode

Layer sequences other than the aforementioned structure are also possible, these being known to those skilled in the art. For example, it is possible that the OLED does not have all the layers mentioned; for example, an OLED having the layers (1) (anode), (3) (light-emitting layer) and (6) (cathode) is likewise suitable, wherein the functions of layers (2) (hole conductor layer) and (4) (blocking layer for holes/excitons) and (5) (electron conductor layer) are assumed by the adjoining layers. OLEDs having layers (1), (2), (3) and (6) or layers (1), (3), (4), (5) and (6) are likewise suitable. In addition, the OLEDs may have a blocking layer for electrons/excitons between the anode (1) and the hole conductor layer (2). The structure of an OLED is shown in schematic form in FIG. 1 (BPhen Cs=bathophenanthroline doped with Cs; HBL=hole-blocking layer, mCP=1,3-bis(N-carbazolyl)-benzene, spiro-TTB=2,2′,7,7′-tetra(N,N-ditolyl)amino-9,9-spirobifluorene, TCTA=tris(4-carbazoyl-9-ylphenyl)amine, F6-TCNNQ=2,2-(perfluoronaphthalene-2,6-diylidene)dimalononitrile, ITO=indium tin oxide, Al=aluminum, 1F4Cz=9,9′,9″,9″′-(2-fluoro-6-(trifluoromethyl)benzo-1,3,4,5-tetrayl)tetrakis(9H-carbazole)).

The compounds of the formula (I) may use as emitter or matrix materials in the light-emitting layer.

The compounds of the formula (I) may be present as the sole emitter and/or matrix material—without further additions—in the light-emitting layer. However, it is likewise possible that, as well as the compounds of the formula (I), further compounds are present in the light-emitting layer. For example, one or more fluorescent dyes may be present in order to alter the emission color of the emitter molecule present. In addition, it is possible to use a diluent material. This diluent material may be a polymer, for example poly(N-vinylcarbazole) or polysilane. However, the diluent material may likewise be a small molecule, for example 4,4′-N,N′-dicarbazolebiphenyl (CBP=CDP) or tertiary aromatic amines.

The individual layers of the OLED among those mentioned above may in turn be formed from 2 or more layers. For example, the hole-transporting layer may be formed from a layer into which holes are injected from the electrode, and a layer which transports the holes away from the hole-injecting layer into the light-emitting layer. The electron-transporting layer may likewise consist of multiple layers, for example a layer in which electrons are injected by the electrode, and a layer which receives electrons from the electron-injecting layer and transports them into the light-emitting layer. These said layers are each selected according to factors such as energy level, thermal resistance and charge carrier mobility, and energy differential of the layers mentioned with the organic layers or the metal electrodes. The person skilled in the art will be able to choose the construction of the OLEDs such that it is optimized for the organic compounds used as emitter substances.

In order to obtain particularly efficient OLEDs, the HOMO (highest occupied molecular orbital) of the hole-transporting layer should be matched to the work function of the anode and the LUMO (lowest unoccupied molecular orbital) of the electron-transporting layer should be matched to the work function of the cathode.

The anode (1) is an electrode which provides positive charge carriers. It may be formed, for example, from materials including a metal, a mixture of various metals, a metal alloy, a metal oxide or a mixture of various metal oxides. Alternatively, the anode may be a conductive polymer. Suitable metals include the metals of groups 11, 4 and 5 of the Periodic Table of the Elements and the transition metals of groups 9 and 10. If the anode is to be transparent, in general, mixed metal oxides of groups 12, 13 and 14 of the Periodic Table of the Elements are used, for example indium tin oxide (ITO). It is likewise possible that the anode (1) comprises an organic material, for example polyaniline, as described, for example, in Nature, Vol. 357, pages 477 to 479 (11 Jun. 1992). At least either the cathode or the anode should be at least partly transparent in order to be able to emit the light formed. The material used for the anode (1) may be ITO.

Suitable hole conductor materials for layer (2) of the OLEDs are disclosed, for example, in Kirk-Othmer Encyclopedia of Chemical Technology, 4th edition, vol. 18, pages 837 to 860, 1996. Both hole-transporting molecules and polymers can be used as hole transport material. Customarily used hole-transporting molecules are selected from the group consisting of tris-[N-(1-naphthyl)-N-(phenylamino)]triphenylamine (1-NaphDATA), 4,4′-bis[N-(1-naphthyl)-N-phenyl-amino]biphenyl (α-NPD), N,N′-diphenyl-N,N′-bis(3-methylphenyl)-[1,1′-biphenyl]-4,4′-diamine (TPD), 1,1-bis[(di-4-tolylamino)phenyl]cyclohexane (TAPC), N,N′-bis(4-methylphenyl)-N,N′-bis(4-ethylphenyl)-[1,1′-(3,3′-dimethyl)biphenyl]-4,4′-diamine (ETPD), tetrakis-(3-methylphenyl)-N,N,N′,N′-2,5-phenylendiamine (PDA), α-phenyl-4-N,N-diphenylaminostyrene (TPS), p-(diethylamino)-benzaldehyde diphenylhydrazone (DEH), triphenylamine (TPA), bis[4-(N,N-diethylamino)-2-methylphenyl)(4-methyl-phenyl)methane (MPMP), 1-phenyl-3-[p-(diethylamino)styryl]-5-[p-(diethylamino)phenyl]pyrazoline (PPR or DEASP), 1,2-trans-bis(9H-carbazol-9-yl)cyclobutane (DCZB), N,N,N′,N′-tetrakis(4-methylphenyl)-(1,1′-biphenyl)-4,4′-diamine (TTB), 4,4′,4″-tris(N,N-diphenylamino)triphenylamine (TDTA), porphyrin compounds and phthalocyanines such as copper phthalocyanines. Customarily used hole-transporting polymers are selected from the group consisting of polyvinylcarbazoles, (phenylmethyl)polysilanes and polyanilines. It is likewise possible to obtain hole-transporting polymers by doping hole-transporting molecules into polymers such as polystyrene and polycarbonate. Suitable hole-transporting molecules are the molecules already mentioned above.

In addition, it is possible in various embodiments to use carbene complexes as hole conductor materials, where the band gap of the at least one hole conductor material is generally greater than the band gap of the emitter material used. In the context of the present application, “band gap” is understood to mean the triplet energy. Suitable carbene complexes are, for example, carbene complexes as described in WO 2005/019373 A2, WO 2006/056418 A2 and WO 2005/113704, and in the prior European applications EP 06112228.9 and EP 06112198.4 that were yet to be published at the priority date of the present application.

The light-emitting layer (3) comprises at least one emitter material. The emitter may in principle be a fluorescence or phosphorescence emitter, suitable emitter materials being known to those skilled in the art. The at least one emitter material may be a phosphorescence emitter. At least one of the emitter materials present in the light-emitting layer (3) here is a compound of the formula (I) as described herein. Furthermore, it is possible to use at least one compound of the formula (I) additionally as matrix material. Alternatively, commonly used matrix materials that are customary in the prior art are known to those skilled in the art. Illustrative matrix materials are selected from the classes of the oligoarylenes (e.g. 2,2′,7,7′-tetraphenylspirobifluorene or dinaphthylanthracene), especially the oligoarylenes containing fused aromatic groups, for example anthracene, benzanthracene, benzphenanthrene, phenanthrene, tetracene, coronene, chrysene, fluorene, spirofluorene, perylene, phthaloperylene, naphthaloperylene, decacyclene, rubrene, the oligoarylenevinylenes (e.g. DPVBi=4,4′-bis(2,2-diphenylethenyl)-1,r-biphenyl) or spiro-DPVBi according to EP 676461), or the polypodal metal complexes, especially metal complexes of 8-hydroxyquinoline, e.g. AIQ3 (=aluminum(111) tris(8-hydroxyquinoline)) or bis(2-methyl-8-quinolinolato)-4-(phenylphenolinolato)aluminum. In general, suitable matrix materials are known to the person skilled in the art, for example, from Organic Light-Emitting Materials and Devices (Optical Science and Engineering Series; Ed.: Z. Li, H. Meng, CRC Press Inc., published 2006).

The blocking layer for holes/excitons (4) may include hole blocker materials typically used in OLEDs, such as 2,9-dimethyl-4,7-diphenyl-1,10-phenanthroline (bathocuproin, (BCP)), bis-(2-methyl-8-quinolinato)-4-phenylphenylato)aluminum(111) (BAIq), phenothiazine S,S-dioxide derivatives and 1,3,5-tris(N-phenyl-2-benzylimidazol)benzene) (TPBI), and TPBI and BAIq are also suitable as electron-conducting materials. In a further embodiment, it is possible to use compounds containing aromatic or heteroaromatic rings bonded via groups containing carbonyl groups, as disclosed in WO2006/100298, as blocking layer for holes/excitons (4) or as matrix materials in the light-emitting layer (3).

In various embodiments, an OLED comprising the following layers: (1) anode, (2) hole conductor layer, (3) light-emitting layer, (4) blocking layer for holes/excitons, (5) electron conductor layer and (6) cathode, and optionally further layers, where the light-emitting layer (3) comprises at least one compound of the formula (I).

Suitable electron conductor materials for the layer (5) of the OLEDs include metals chelated to oxinoid compounds, such as tris(8-quinolinolato)aluminum (Alqβ), bis-(2-methyl-8-quinolinato)-4-phenylphenylato)aluminum(111) (BAIq), phenanthroline-based compounds such as 2,9-dimethyl-4,7-diphenyl-1,10-phenanthroline (DDPA=BCP) or 4,7-diphenyl-1,10-phenanthroline (DPA) and azole compounds such as 2-(4-biphenylyl)-5-(4-t-butylphenyl)-1,3,4-oxadiazole (PBD) and 3-(4-biphenylyl)-4-phenyl-5-(4-t-butylphenyl)-1,2,4-triazole (TAZ) and 2,2′,2″-(1,3,5-phenylene)tris-[1-phenyl-1H-benzimidazole] (TPBI). It is possible here for the layer (5) to serve both to facilitate electron transport and as a buffer layer or as a barrier layer in order to prevent quenching of the exciton at the interfaces of the layers of the OLED. The layer (5) may improve the mobility of the electrons and reduces quenching of the exciton. Electron conductor materials suitable are TPBI and BAIq.

Some of the materials mentioned above as hole conductor materials and electron conductor materials can fulfill multiple functions. For example, some of the electron-conducting materials are simultaneously hole-blocking materials if they have a low-lying HOMO. These may be used, for example, in the blocking layer for holes/excitons (4). However, it is likewise possible that the function as hole/excitons blocker is assumed by the layer (5), such that the layer (4) can be dispensed with.

The charge transport layers may also be electronically doped in order to improve the transport properties of the materials used, in order firstly to make the layer thicknesses more generous (avoidance of pinholes/short-circuits) and secondly to minimize the operating voltage of the device. For example, the hole conductor materials can be doped with electron acceptors; for example, phthalocyanines or arylamines such as TPD or TDTA can be doped with tetrafluoro-tetracyanoquinodimethane (F4-TCNQ). The electron conductor materials can be doped, for example, with alkali metals, for example AIq3 with lithium. Electronic doping is known to those skilled in the art and is disclosed, for example, in W. Gao, A. Kahn, J. Appl. Phys., vol. 94, no. 1, 1 Jul. 2003 (p-doped organic layers); A. G. Werner, F. Li, K. Harada, M. Pfeiffer, T. Fritz, K. Leo. Appl. Phys. Lett., vol. 82, no. 25, 23 Jun. 2003 and Pfeiffer et al., Organic Electronics 2003, 4, 89-103.

The cathode (6) is an electrode that serves to introduce electrons or negative charge carriers. Suitable materials for the cathode are selected from the group consisting of alkali metals of groups Ia, for example Li, Cs, alkaline earth metals of group IIa, for example calcium, barium or magnesium, metals of group IIb of the Periodic Table of the Elements (old ILJPAC version), comprising the lanthanides and actinides, for example samarium. In addition, it is also possible to use metals such as aluminum or indium, and combinations of all the metals mentioned. In addition, lithium-containing organometallic compounds or LiF may be applied between the organic layer and the cathode in order to reduce the operating voltage.

The OLED may additionally comprise further layers that are known to those skilled in the art. For example, a layer that facilitates the transport of the positive charge and/or adjusts the band gap of the layers with respect to one another may be applied between the layer (2) and the light-emitting layer (3). Alternatively, this further layer may serve as a protective layer. In an analogous manner, it is possible for additional layers to be present between the light-emitting layer (3) and the layer (4), in order to facilitate the transport of the negative charge and/or to adjust the band gap between the layers relative to one another. Alternatively, this layer can serve as protective layer.

In various embodiments, the OLED, in addition to layers (1) to (6), comprises at least one of the following further layers:

A hole injection layer between the anode (1) and the hole-transporting layer (2); a blocking layer for electrons between the hole-transporting layer (2) and the light-emitting layer (3); an electron injection layer between the electron-transporting layer (5) and the cathode (6).

The person skilled in the art knows how suitable materials have to be chosen (for example on the basis of electrochemical studies). Suitable materials for the individual layers are known to those skilled in the art and are disclosed, for example, in WO 00/70655.

In addition, it is possible that some or all of the layers used in the OLED have been surface-treated in order to increase the efficiency of charge carrier transport. The selection of the materials for each of the layers mentioned is determined so as to obtain an OLED having a high efficiency and lifetime.

The OLED can be produced by methods known to those skilled in the art. In general, the OLED is produced by successive vapor deposition of the individual layers onto a suitable substrate. Suitable substrates are, for example, glass, inorganic semiconductors or polymer films. Vapor deposition can be accomplished using customary techniques such as thermal evaporation, chemical vapor deposition (CVD), physical vapor deposition (PVD) and others. In an alternative process, the organic layers of the OLED can be applied from solutions or dispersions in suitable solvents, employing coating techniques known to those skilled in the art.

In general, the various layers have the following thicknesses: anode (1) 50 to 500 nm, alternatively 100 to 200 nm; hole-conducting layer (2) 5 to 100 nm, alternatively 20 to 80 nm, light-emitting layer (3) 1 to 100 nm, alternatively 10 to 80 nm, blocking layer for holes/excitons (4) 2 to 100 nm, alternatively 5 to 50 nm, electron-conducting layer (5) 5 to 100 nm, alternatively 20 to 80 nm, cathode (6) 20 to 1000 nm, alternatively 30 to 500 nm. The relative position of the recombination zone of holes and electrons in the OLED in relation to the cathode and hence the emission spectrum of the OLED can be affected by factors including the relative thickness of each layer. This means that the thickness of the electron transport layer should be chosen such that the position of the recombination zone is matched to the optical resonator property of the diode and hence to the emission wavelength of the emitter. The ratio of the layer thicknesses of the individual layers in the OLED is dependent on the materials used. The layer thicknesses of any additional layers used are known to those skilled in the art. It is possible that the electron-conducting layer and/or the hole-conducting layer have greater thicknesses than the layer thicknesses specified when they are electrically doped.

The light-emitting layer and/or at least one of the further layers that are optionally present in the OLED comprises at least one compound of the general formula (I). While the at least one compound of the general formula (I) is present as emitter material and/or matrix material in the light-emitting layer, the at least one compound of the general formula (I) may be used in the at least one further layer of the OLED, in each case alone or together with at least one of the further materials mentioned above that are suitable for the corresponding layers. It is likewise possible that the light-emitting layer comprises one or more further emitter and/or matrix materials as well as the compound of the formula (I).

The efficiency of the OLEDs can be improved, for example by optimizing the individual layers. For example, it is possible to use high-efficiency cathodes such as Ca or Ba, optionally in combination with an intermediate layer of LiF. Shaped substrates and novel hole-transporting materials that bring about a reduction in the operating voltage or an increase in the quantum efficiency are likewise usable in the OLEDs. In addition, additional layers may be present in the OLEDs in order to adjust the energy level of the various layers and in order to facilitate electroluminescence.

The OLEDs can be used in all devices in which electroluminescence is useful. Suitable devices may be selected from stationary and mobile display screens and lighting units. Stationary display screens are, for example, screens of computers, televisions, screens in printers, kitchen appliances and advertising panels, lighting units and information panels. Mobile display screens are, for example, screens in mobile phones, laptops, digital cameras, motor vehicles, and destination displays on buses and trains.

In addition, it is possible to use the compounds of the formula (I) in various embodiments in OLEDs with inverse structure. The construction of inverse OLEDs and the materials that are typically used therein are known to those skilled in the art.

All the documents cited are incorporated herein in their entirety by reference. Further embodiments can be found in the examples which follow, but the invention is not restricted thereto.

It will be apparent and is the intention that all embodiments disclosed herein in connection with the compounds described are equally applicable to the uses and methods described, and vice versa. Embodiments of this kind are therefore likewise covered by the scope of the present invention.

### Examples

### 9-(2,3,5,6-Tetrafluoro-4-(trifluoromethyl)phenyl)-9H-carbazole (4F1 Cz)

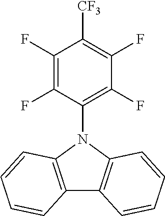 [SMILES: Fc1c(F)c(C(F)(F)F)c(F)c(F)c1-n1c2ccccc2c2ccccc21]

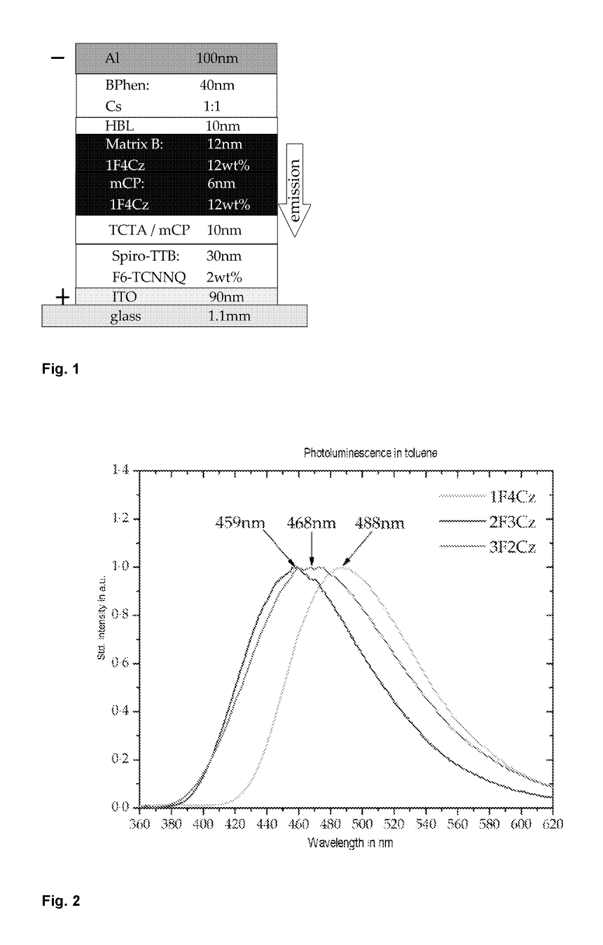

The product was obtained by reaction otoctafluorotoluene (1.41 g; 5.98 mmol) with carbazole (1 g; 7.2 mmol) in the presence of potassium carbonate (0.99 g; 7.2 mmol) in 10 ml of dry DMF (dimethylformamide). The reaction mixture was stirred at room temperature over a period of 26 hours. The target substance was purified by column chromatography and obtained with a yield of 86% (1.46 g). The photoluminescence in toluene was determined and is shown in FIG. 2.

### 9-(2,4,6-Trichloro-3,5-difluorophenyl)-9H-carbazole

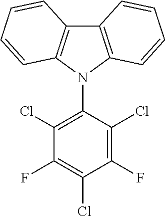 [SMILES: Fc1c(Cl)c(F)c(Cl)c(-n2c3ccccc3c3ccccc32)c1Cl]

The product was obtained by reaction of 1,3,5-trichloro-2,4,6-trifluorobenzole (1.98 g; 14.4 mmol) with carbazole (2 g; 11.98 mmol) in the presence of potassium carbonate (3.3 g; 23.9 mmol) in 10 ml of dry DMSO (dimethyl sulfoxide). The reaction mixture was stirred over a period of 7 hours and with heating to 50° C. The target substance was purified by column chromatography and obtained with a yield of 13% (0.56 g).

### 4-(9H-Carbazol-9-yl)-3-(trifluoromethyl)benzonitrile

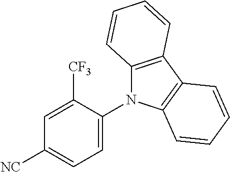 [SMILES: N#Cc1ccc(-n2c3ccccc3c3ccccc32)c(C(F)(F)F)c1]

The product was obtained by reaction of 4-fluoro-3-(trifluoromethyl)benzonitrile (1 g; 5.2 mmol) with carbazole (0.88 g; 5.2 mmol) in the presence of potassium carbonate (1.44 g; 10.4 mmol) in 10 ml of dry DMF (dimethylformamide). The reaction mixture was stirred over a period of 20 hours and with heating to 80° C. The target substance was purified by means of recrystallization from an ether/hexane mixture and obtained with a yield of 47% (0.8 g).

### 9-(3-Carbazol-9-yl-2,4,6-trichloro-5-fluorophenyl)carbazole

The product was obtained by reaction of 1,3,5-trichloro-2,4,6-trifluorobenzene

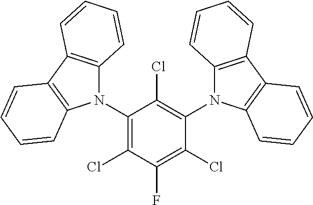 [SMILES: Fc1c(Cl)c(-n2c3ccccc3c3ccccc32)c(Cl)c(-n2c3ccccc3c3ccccc32)c1Cl]

(1.98 g; 14.4 mmol) with carbazole (2 g; 11.98 mmol) in the presence of potassium carbonate (3.3 g; 23.9 mmol) in 10 ml of dry DMSO (dimethyl sulfoxide). The reaction mixture was stirred over a period of 7 hours and with heating to 50° C. The target substance was purified by column chromatography and obtained with a yield of 52% (1.63 g).

### 9,9′-(2,4,5-Trifluoro-6-(trifluoromethyl)-1,3-phenylene)bis(9H-carbazole) (3F2Cz)

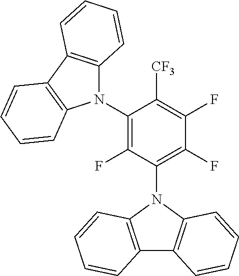 [SMILES: Fc1c(F)c(C(F)(F)F)c(-n2c3ccccc3c3ccccc32)c(F)c1-n1c2ccccc2c2ccccc21]

The product was obtained by reaction of octafluorotoluene (1.5 g; 6.3 mmol) with carbazole (2.12 g; 12.7 mmol) in the presence of potassium carbonate (1.93 g; 13.9 mmol) in 10 ml of dry DMF (dimethylformamide). The reaction mixture was stirred at room temperature over a period of 24 hours. The target substance was purified by column chromatography and obtained with a yield of 53% (1.76 g). The photoluminescence in toluene was determined and is shown in FIG. 2.

### 9,9′,9″-(2,4-Difluoro-6-(trifluoromethyl)benzo-1,3,5-triyl)tris(9H-carbazole) (2F3 Cz)

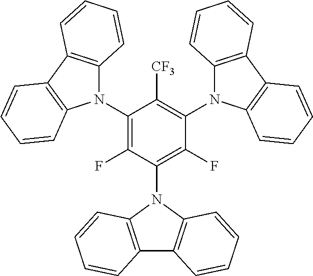 [SMILES: Fc1c(-n2c3ccccc3c3ccccc32)c(F)c(-n2c3ccccc3c3ccccc32)c(C(F)(F)F)c1-n1c2ccccc2c2ccccc21]

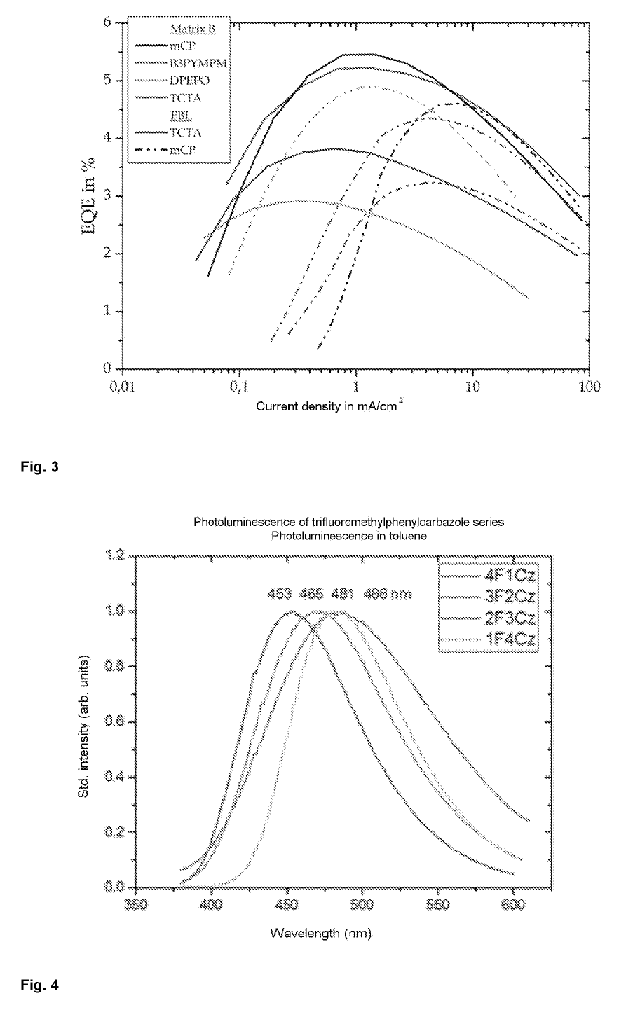

The product was obtained by reaction of octafluorotoluene (1.5 g; 6.3 mmol) with carbazole (2.83 g; 16.9 mmol) in the presence of potassium hydroxide (1.93 g; 33.9 mmol) in 35 ml of dry acetone. The reaction mixture was heated to reflux over a period of one hour. The target substance was purified by column chromatography and obtained with a yield of 53% (2.42 g). The photoluminescence in toluene was determined and is shown in FIG. 2. 1F4Cz in FIG. 2 denotes 9,9′,9″,9″′-(2-fluoro-6-(trifluoromethyl)benzo-1,3,4,5-tetrayl)tetrakis(9H-carbazole) (1F4Cz). For the compound 1F4Cz, FIG. 3 also shows the EQE in % in various host materials: B3PYMPM=bis-4,6-(3,5-di-3-pyridylphenyl)-2-methylpyrimidine, mCP=1,3-bis-(N-carbazoyl)benzene, TCTA=tris(4-carbazoyl-9-ylphenyl)amine).

1. A compound of the general formula (I) 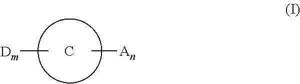
where
C denotes an aromatic or heteroaromatic, nitrogen-containing 6-membered carbon ring selected from the group consisting of benzene, pyridine, pyrimidine, pyridazine, triazine and pyrazine;
the substituent D at each instance independently denotes a nitrogen-containing heteroaryl radical bonded to the aromatic system C via a nitrogen atom, or a radical of the formula —N(Ar)₂, —N(HetAr)(Ar), —N(HetAr)₂, where each Ar is independently an aryl radical and each HetAr is independently a heteroaryl radical;
the substituent A at each instance independently denotes electron acceptors selected from the group consisting of C1-C12 perfluoroalkyl, especially CF₃, Cl, F, Br, SCN or CN;
m denotes an integer selected from 1, 2, 3, 4 and 5;
n denotes an integer selected from 1, 2, 3, 4 and 5;
where 3≦m+n≦6;
and where the substituents D and A are each bonded to the aromatic system C,
wherein not more than one substituent A denotes CN.

2. A compound according to claim 1, where the compound is a compound of the formula (Ia) 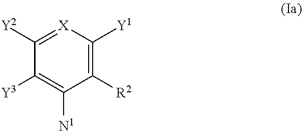
where
X is C—CF₃, C—Cl, C—F, C—CN, or N;
R² is F, Cl, Br, C1-C12 perfluoroalkyl;
N¹ is a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical;
Y¹, Y² and Y³ are each independently F, Cl, Br, C1-C12 perfluoroalkyl, CF₃, CCl₃, SCN, CN, H, a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical;
wherein
i. only one of the substituents Y¹⁻³ and R² is CN;
ii. when X is C—CN, none of the substituents Y¹⁻³ and R² is CN;
iii. when X is C—F, C—Cl or C—CN, and Y¹ and Y² are each independently a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical, Y³ is not F, Cl, Br, or CN.

3. A compound according to claim 1, where the compound is a compound of the formula (II) 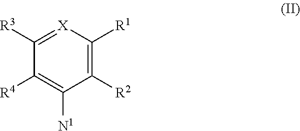
where
X is C—CF₃, C—CCl₃, C—Cl, C—F, C—SCN, C—CN, or N;
N¹ is a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical; and
R¹, R², R³ and R⁴ are each independently F, Cl, Br, CF₃, SCN, CN or H;
wherein
i. only one of the substituents R¹⁻⁴ is CN;
ii. when X is C—CN, none of the substituents R¹⁻⁴ is CN.

4. A compound according to claim 1, where the compound is a compound of the formula (III) 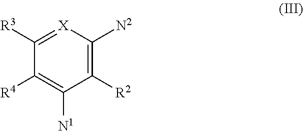
where
X is C—CF₃, C—SCN, C—CCl₃, C—Cl, C—F, C—CN, or N;
N¹ and N² are each independently a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical; and
R², R³ and R⁴ are each independently F, Cl, Br, CCl₃, C1-C12 perfluoroalkyl, especially CF₃, CN or H;
wherein
i. only one of the substituents R^{2,3,4} is CN;
ii. when X is C—CN, none of the substituents R^{2,3,4} is CN.

5. A compound according to claim 1, where the compound is a compound of the formula (IV) 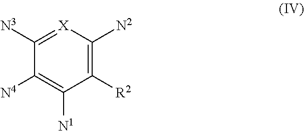
where
X is C—CF₃, C—Cl, C—F, C—SCN, C—CCl₃, C—CN, or N;
R² is F, Cl, Br, SCN, CF₃, CN or H; and
N¹, N², N³ and N⁴ are each independently a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical;
wherein when X is C—CN, R² is not CN.

6. A compound according to claim 1, where the compound is a compound having a the formula from one of (Va)-(Vd): 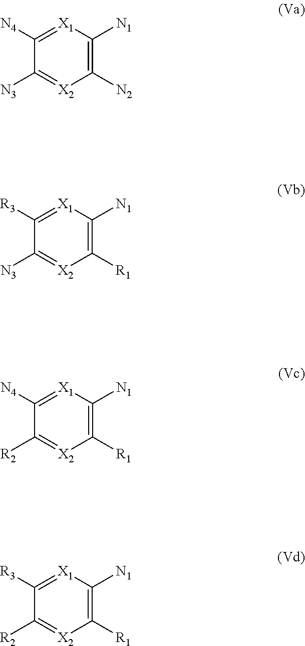
where,
(a) in the compounds of the formula (Va),
X¹ and X² are each independently C—CF₃, C—SCN, C—Cl, C—F, C—CN, or N; and
N¹, N², N³ and N⁴ are each independently a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical,
wherein when X¹ is C—CN, C—F or C—Cl, X² is not C—CN;
(b) in the compounds of the formula (Vb),
X¹ and X² are each independently C—CF₃, C—SCN, C—Cl, C—F, C—CN, or N;
N¹ and N³ are each independently a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical; and
R¹ and R³ are each independently H or F;
(c) in the compounds of the formula (Vc),
X¹ and X² are each independently C—CF₃, C—SCN, C—Cl, C—F, C—CN, or N;
N¹ and N⁴ are each independently a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical,
R¹ and R² are each independently H or F;
(d) in the compounds of the formula (Vd),
X¹ and X² are each independently C—CF₃, C—SCN, C—Cl, C—F, C—CN, or N;
N¹ is a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical; and
R¹, R² and R³ are each independently H or F.

7. A compound according to claim 1, where the compound is a compound having a the formula from one of (VIa)-(VIc) 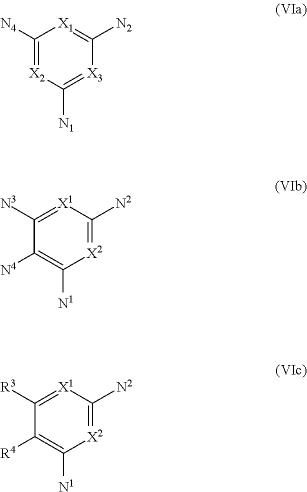
where,
(a) in the compounds of the formula (VIa),
X¹, X² and X³ are each independently C—CF₃, C—CCl₃, C—Cl, C—F, C—CN, or N,
N¹, N² and N⁴ are each independently a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical;
wherein
i. X¹, X² and X³ are not all simultaneously C—CN, C—Cl, C—F or N;
ii. when X¹ and X² are each C—CN, X³ is C—CF₃ or N;
b) in the compounds of the formula (VIb),
X¹ and X² are each N;
N¹, N², N³ and N⁴ are each independently a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical;
c) in the compounds of the formula (VIc),
X¹ is C—CN or N;
X² is N;
N¹ and N² are each independently a nitrogen-containing heteroaryl radical bonded via a nitrogen atom, or a radical of the formula —N(Ar)₂, —N(HetAr)(Ar), —N(HetAr)₂, where each Ar is independently an aryl radical and each HetAr is independently a heteroaryl radical; and
R³ and R⁴ are each independently H or F.

8. Use of at least one compound according to claim 1 in an optoelectronic component from the group comprising: an organic electroluminescent device (OLED), an organic integrated circuit (O-IC), an organic field-effect transistor (O-FET), an organic thin-film transistor (O-TFT), an organic light-emitting transistor (O-LET), an organic solar cell (O-SC), an organic optical detector, an organic photoreceptor, an organic field-quench device (O-FQD), a light-emitting electrochemical cell (LEC), or an organic laser diode (O-laser).

9. An optoelectronic component comprising at least one compound according to claim 1.
